(19) United States
(12) Patent Application Publication
(10) Pub. No.: US 2008/0118522 A1
(43) Pub. Date: May 22, 2008

(54) Naturally occuring IgM antibodies that bind lymphocytes

(76) Inventor: Peter I. Lobo, Charlottesville, VA (US)

Correspondence Address: Peter I. Lobo, 348 Keywest Dr, Charlottesville, VA, 22911, US

(21) Appl. No.: 12/009,912

(22) Filed: Jan. 23, 2008

Related U.S. Application Data
(63) Continuation-in-part of application No. 12/008,778, filed on Jan. 14, 2008.
Application No. 12/008,778 is a continuation-in-part of application No. 11/139,566, filed on May 31, 2005.
Application No. 11/139,566 is a continuation-in-part of application No. 10/292,002, filed on Nov. 7, 2002, now abandoned.
Application No. 10/292,002 is a continuation-in-part of application No. 09/684,813, filed on Oct. 10, 2000, now Pat. No. 6,610,834.
Application No. 09/684,813 is a continuation-in-part of application No. 09/439,690, filed on Nov. 15, 1999, now abandoned.
(60) Provisional application No. 60/108,937, filed on Nov. 18, 1998.

(51) Int. Cl. A61K 39/395; A61P 35/00; A61K 35/12; C12P 21/00; G01N 33/566; G01N 33/53; C07K 16/24; A61P 29/00

(52) U.S. Cl. 424/172.1; 424/93.7; 530/389.1; 435/70.3; 435/7.24; 436/501

(57) ABSTRACT

Human and animal serum contains naturally occurring autoantibodies that develop at birth in absence of deliberate immunization. These antibodies are predominantly of IgM isotype but can include all immunoglobulin isotypes such as IgD, IgA and IgG. Here we describe IgM anti-lymphocyte autoantibodies (IgM-ALA) and show that these antibodies are heterogenous with some antibodies binding to chemokine receptors such as CCR5 and CXCR4 and others binding to other lymphocyte receptors including CD3, CD2, CD4 and CD81. These IgM-ALA, unlike IgG antibodies, are not cytolytic to cells at 37 C and hence function to alter lymphocyte function including cytokine production and act as “blocking antibodies to inhibit binding of chemokines and viruses including HIV-1 and Hepatitis C. IgM antibodies that bind to receptors on lymphocyte also bind to the same or similar class of receptors on other leucocytes and other cells such as cancer cells and endothelial cells. The inventor claims that naturally occurring anti-lymphocyte antibodies inhibit viral infections, cancer and several inflammatory states by binding to chemokine receptors and other cell membrane receptors that activate cells or promote viral entry and replication. Inventor also claims methods for quantitating levels of IgM-ALA with different receptor specificities to aid in preventing disease progression and also claims methods to enhance in-vivo or in-vitro production of IgM-ALA.

This patent application is a continuation in part (CIP) of application Ser. No. 12/008,778 filed on Jan. 14, 2008, now abandoned which is a CIP of U.S. patent application Ser. No. 11/139,566 filed on May 31, 2005, which is a CIP of U.S. patent application Ser. No. 10/292,002, filed Nov. 7, 2002, now abandoned which is a CIP of U.S. patent application Ser. No. 09/684,813 filed Oct. 10, 2000, now U.S. Pat. No. 6,610,834.

### BACKGROUND ART

1. Field of the Invention

The present invention relates generally to naturally occurring IgM anti-lymphocyte antibodies and, more particularly, to a method of inhibiting disease progression through use of these antibodies.

2. Discussion of the Background

In inventors prior art (application Ser. No. 09/684,813 filed on Oct. 10, 2000 now U.S. Pat. No. 6,610,834) inventor shows that IgM anti-lymphocyte autoantibodies (IgM-ALA) bind to chemokine receptors and through this mechanism inhibits HIV-1 from infecting cells. In patent application Ser. No. 11/139,566 filed on May 31, 2005, the inventor shows that IgM-ALA, in addition, binds to CD3 and CD4 and in the description shows that (i) IgM-ALA inhibits activation and proliferation of T cells and production of cytokines by leucocytes and through this mechanism IgM-ALA has an inhibitory effect on T cell mediated inflammatory processes as well as on inhibiting HIV-1 infectivity (ii) IgM-ALA enhance apoptosis of malignant cells and (iii) IgM inhibits HIV-1 in an in-vivo animal model. In this present application inventor shows that IgM-ALA are clearly naturally occurring, inhibit in-vivo allograft rejection i.e. an inflammatory state and in addition shows that certain IgM-ALA bind to CD81, and that IgM inhibits hepatitis C from infecting cells. In addition, the inventor shows that in-vivo LPS enhances IgM-ALA in normal mice without producing auto-immune disease or enhancing T cell activation. In the description, inventor provides evidence to show why IgM-ALA is a new invention that is different or distinct from IVIG and other IgG anti-lymphocyte receptor antibodies (e.g. anti-CD4 or anti-CXCR4). Below, inventor provides a detailed background information of the above embodiments.

Normal humans and animals have naturally occurring auto-antibodies (referred to as NAA), which are produced in the absence of deliberate immunization with the target antigen. The self-reactive repertoire of NAA are restricted to a small number of self-reactive antigens that are markedly conserved among individuals. NAA can also bind to non-self antigens, which have the same or similar antigenic specificity as the autoantigen. Some of these NAA can be detected at birth, but the full repertoire of NAA develops later in life, usually by early childhood. Prior art has clearly demonstrated that NAA are mostly polyreactive in that a single monoclonal NAA can recognize several closely similar self antigens, which possess a unique but distinct set of epitope specificities. The nature of this polyreactivity is best exemplified by rheumatoid factor, which is an IgM NAA that recognizes and binds to the Fc region of different self and non-self IgG including the different IgG subclasses, but does not bind to other glycoproteins or self nucleo-proteins. The antigen binding domain of NAA (i.e. variable region of antibody) are in general encoded by germline genes, which are subjected to no or minimal mutation and this characteristic is responsible for the polyreactivity and low affinity binding of these antibodies. Conversely, genes encoding the variable region of antibodies produced in response to a foreign antigen or autoantibodies that cause disease (e.g. thyroiditis) are hypermutated and this genetic characteristic renders these antibodies highly specific with high binding affinity. Hence, the presence of NAA at birth and in normal sera together with polyreactivity and low binding affinity, resulting form their genetic makeup, distinguishes these antibodies from the conventional antibodies produced after deliberate immunization or disease producing autoantibodies. Prior art teaches that NAA are predominantly of the IgM isotype but NAA of other isotypes have also been described (see Nakamura M, J of Immunol. 1988, vol 141, p 4165-72). Prior art has used antibodies, typically produced after immunization, and with high binding affinity and with high specificity, to protect against infections or to inhibit immune mediated disorders. The current invention is novel in that the antibodies used are naturally occurring. More information on NAA are reviewed in Lacroix-Desmazes S. et. al, J of Immunological Methods 216:117-137, 1998 and Cervenak J, Acta Microbiologica et Immunologica Hungaria 46:53-62, 1999.

Several mechanisms have been proposed for the selective production of NAA and include (i) positive selection by conserved self-ligands (ii) genetic programming and (iii) response to cross-reactive non-self antigens. However, the presence of NAA in umbilical cord blood and the finding of restricted and conserved NAA repertoires in normal individuals would lend support for the first two mechanisms under physiological conditions (See Stahl D, et. al J of Immunol Methods Vol. 240, p. 1-14, 2000 for more information in this regard). Autoantibodies that arise in response to cross-reactive non-self antigens e.g. antigens from infectious agents, are in general of the IgG isotype and are implicated in causing auto-immune disease.

Prior art has shown that NAA are produced by a small subset of B cells identified as B-1 cells, which express IgM and CD5 on their cell membrane. Antibodies to non-self antigens are produced by the B-2 subset of B cells which express IgM (but not CD5) on their cell membrane. These B-1 cells are found in the bone-marrow and spleen of humans. B-1 cells, unlike B-2 cells, fail to produce antigen specific antibodies in response to non-self antigens and in addition do not proliferate or enhance production of IgM-NAA after cross-linking membrane expressed IgM. However B-1 cells enhance production of IgM-NAA after stimulation with mitogens (e.g. LPS), phorbol esters, and cytokines (e.g. IL-4, IL-5). See Baumgarth N, et. al, Springer Semin Immun Vol. 26, P. 347-362, 2005 for a detailed review of this subject. Prior art also shows that B-1 cells proliferate in the presence of self-antigens (See Julien S, et. al J of Immunol, Vol 169, p. 4198-4204, 2002).

There is no prior art to directly show that autoantibodies produced by B-1 cells can cause auto-immune diseases in humans or animal models that lack a genetic predisposition to auto-immunity. Prior art teaches that the onset of autoimmune diseases correlates with a switch from production of IgM to IgG autoantibodies (See Naparstek and Plotz, Annual Rev Immunol, Vol 11, p. 79-104, 1993). Even in auto-immune prone mice, the role of B-1 cells in causing auto-immune disease remains controversial especially since B-1 secreted antibodies have low affinity binding. (See Murakami and Honjo, Immunol Today, Vol. 16, p. 534-539, 1995 and Boes M, Molecular Immunol, Vol. 37, p. 1141-1149, 2000). Murakami teaches that in the development of SLE, B-1 cell derived antibodies against single stranded (ss) DNA are less pathogeneic than IgGs specific for double stranded (ds) DNA, which are generally assumed to be produced by conventional B cells . . . the question remains open as to whether B-1 cells produce the pathogenic autoantibodies that are involved in auto-immune disease. Boes teaches that IgM autoantibodies produced naturally or as part of an auto-immune response (e.g. in MLR/Lpr mice) may lessen the severity of auto-immune pathology associated with IgG autoantibodies. The findings in the current invention could provide an explanation supporting the teaching of Boes. George J et. al (See Human antibodies Vol. 8, p. 70-75, 1997) teaches that natural autoantibodies (NAA) i.e. IgM anti-(ss) DNA (16/6) and anti-cardiolipin antibodies, found in SLE patients and in patients with primary anti-phospholipid syndrome (PAPS), can cause disease in mice. However, George in his teaching indicate that the disease in mice could result from generation of anti-idiotypic antibodies in response to IgM-NAA and not from a direct effect of IgM-NAA especially since in mice, the disease occurred several weeks after administration of IgM-NAA antibodies.

Normal human and animals have in their blood low levels of circulating naturally occurring IgM antibodies that bind to their own leukocytes such as, for example, B and T lymphocytes, without causing cell lysis at 37° C. Hence such antibodies bind to cell receptors at 37° C. and operate to block receptors as well as alter cell function. Such IgM antibodies are, therefore, referred to as “IgM anti-lymphocyte autoantibodies.” These IgM anti-lymphocyte antibodies are present at birth and in normal sera (See Lobo P I, Transplanatation, Vol. 32, p. 233-237, 1981 and Cervenak J et. al. Acta Microbiologica et Immunologica Hungarica Vol. 46, P. 53-62, 1999). These IgM anti-lymphocyte antibodies bind to membrane receptors present on lymphocytes. These lymphocyte membrane receptors are also expressed on the membrane of cells from other organs. For example chemokine receptors on lymphocytes are also expressed on fibrocytes, dendritic cells and other bone marrow precursor cells. CD81 receptors are expressed on lymphocytes and other cells including hepatocytes. Similarly most malignant cells including leukemia cells, breast, colon and lung cells express chemokine receptors, which play a role in the growth and metastatic spread of these cancer cells. CD4 is expressed on T lymphocytes and many other cells including B cells, dendritic cells, macrophages and brain cells. Hence IgM anti-lymphocyte NAA can bind to macrophages, neutrophils, endothelial cells, hepatocytes, malignant cells and other cells and furthermore bind to allogenic cells in addition to autologous leukocytes. Both, animal IgM anti-lymphocyte NAA (for example mouse, rat, goat, horse, rabbit) and human IgM anti-lymphocyte NAA bind to the same human cells. Hence, in this application, IgM anti-lymphocyte auto-antibodies (whether human or animal) will be referred to as IgM anti-lymphocyte or leucocyte antibodies or autoantibodies, (IgM-ALA) and autoantibodies or antibodies will be used interchangeably. Prior art also teaches us that naturally occurring IgM-ALA are heterogeneous comprising several different clones of IgM, each with a different specificity, but like other NAA's, each of these IgM clones can be polyreactive and therefore can bind to the same or similar, class of receptors. For example, prior art has shown that IgM Rheumatoid factor, like other NAA, are polyreactive and will therefore bind to self and non-self IgG as well as all subclasses of IgG. The inventor shows that a monoclonal IgM isolated in his laboratory e.g., CK15 binds to CCR5, CCR3 and CCR1 but not CXCR4 and thus IgM anti-chemokine receptor NAA, like IgM Rheumatoid factor, can bind to different chemokine receptors, which are “similar” in that they be long to the same class of C-C receptors.

Prior art show that other inventors have produced IgG antibodies, both monoclonal and polyclonal, that bind to different receptors on human lymphocytes. Such IgG antibodies bind to CD3, IL-2R, CD4 and other cell receptors. These IgG antibodies are not naturally occurring as they are produced after immunization with cell receptors or cells. Also prior art shows that IVIG has IgG antibodies that bind to CD4 on lymphocytes and in this prior art such antibodies are considered naturally occurring as they are also polyreactive. (See Hurez V, et al., Therapeutic Immunol, Vol. 1, p. 269-73, 1994). The inventor believes that IgG anti-CD4 antibodies present in human IVIG preparations are not naturally occurring for three reasons. Firstly. prior art shows that normal adult sera do not contain IgG anti-leucocyte autoantibodies or alloantibodies (See Lobo P I, et. al. Transplant International, Vol. 8, p. 472-480, 1995 and Lobo P I, Transplantation, Vol. 32, p. 233-237, 1981). Lobo teaches that IgG binds to FcR receptors present mostly on B and NK cells and a small subset (<20%) of T cells from peripheral human blood and this binding is mediated by the Fc domain of IgG. Presence of IgG anti-CD4 in normal adult sera should have been readily apparent as IgG from normal sera should have bound to 40-50% of T cells in human peripheral blood but this finding cannot be demonstrated. Secondly, in this application, inventor shows that IgM-ALA but not IgG anti-leucocyte antibodies are present in umbilical cord sera. Thirdly, prior art has shown that IgG antibodies produced after exposure to a foreign antigen can also be polyreactive like IgM-ALA (See Thompson K M et. al, Immunology, Vol. 76, p. 146-157, 1992). Hence, the identification of polyreactivity in IgG antibodies, present in IVIG, is not by itself sufficient proof to show that such antibodies are “naturally occurring”. The inventor believes that the presence of IgG anti-lymphocyte antibodies e.g. IgG-anti CD4, in IVIG results from exposure of some healthy donors to cross-reactive non-self antigens.

There is other prior art that relates to current invention (See Metlas R, et al Current HIV Res: Vol. 5, p. 261-265, 2007; Rodman T C, U.S. Pat. Nos. 7,189,826 and 5,606,026 and Rodriguez M et al. US Patent 2003/0185827). These prior art are distinct from the instant invention for the following reasons. Metlas teaches that normal human serum has naturally occurring immunoglobulins (of all isotypes) that binds to IgG covalently linked to Sepharose, and that these antibodies with anti-IgG specificity also inhibit some (but not all) HIV-1 isolates in an in-vitro assay using human PBL. In the current application, inventor claims that naturally occurring IgM with specificity to lymphocyte receptors (i.e. HIV-1 coreceptors) inhibits HIV-1 and therefore the current invention is distinct from the prior art of Metlas. Rodman T C teaches that naturally occurring IgM antibodies (especially IgM anti-Tat) in IVIG preparations is inhibitory to HIV-1. The inventor objects to the teachings of Rodman as prior art shows that IVIG contains less than 2% IgM, usually trace amounts (See Romer J et al, Vox Sanguinis, Vol. 42, p. 62-73, 1982). Secondly Rodman provides no prior art to show that IgM anti-Tat inhibits HIV-1 infectivity of cells. Rodriguez teaches that naturally occurring polyclonal and monoclonal IgM, having specificity for structures and cells in the central nervous system (CNS) particularly oligodendrocytes, can be used to treat demyelinating disorders of CNS. Rodriguez also teaches that the inflammatory process seen in the demyelinating disorders may not be T cell mediated and is most likely secondary to an unidentified environmental factor (e.g. virus) that precipitates the inflammatory response in the CNS (See p. 1 of Rodriguez's patent). This prior art of Rodriguez is distinct from the current application as the inventor claims that naturally occurring IgM with specificity for lymphocyte receptors, including T cell receptors, has anti-inflammatory effects, especially for inflammatory states mediated by T cells.

Production of IgM-ALA by B-1 cells is enhanced in inflammatory states (e.g. patients on chronic dialysis, chronic lung inflammation, sarcoidosis, bowel inflammation), autoimmune disorders (e.g. SLE) and in infectious diseases such as, for example, HIV-1, malaria, Epstein-Barr virus (“EBV”) and cytomegalovirus (“CMV”) and viral hepatitis including hepatitis C and B. Individuals with asymptomatic HIV-1, therefore, have high levels of IgM anti-leukocyte autoantibodies. There is no prior art demonstrating that the enhanced production of IgM-ALA in these various disease states is associated with worsening of the disease state or is a cause of the immune dysfunction seen in these various disorders. From teachings of prior art inventor believes that enhanced production of IgM-ALA in these infective/inflammatory states results from stimulation of B-1 cells by non-self antigens with a mitogenic effect (e.g. LPS) or release of cytokines.

The inventor's studies show, however, that chemokine receptors are one of the cell membrane receptors that bind to these IgM-ALA autoantibodies. The inventor shows that IgM inhibits binding of chemokines to their receptors, inhibits chemokine induced internalization of the chemokine receptor, and inhibits chemotaxis of normal leucocytes and malignant cells and through these mechanisms, the inventor believes that naturally occurring IgM-ALA inhibit the inflammatory processes and spread of malignant cells.

The inventor's studies also show that IgM autoantibodies that bind to lymphocyte receptors are heterogeneous and show that IgM-ALA binds to the CD3 and CD4 receptor on T cells and in addition, downregulates CD2 and CD4 on T cells and CD80 and CD86 on macrophages. Accordingly the inventor shows that IgM-ALA, by binding to CD3 and CD4 and by down regulating CD2, CD80 and CD86 inhibits T cell activation, cytokine production e.g., IL-13 and TNF-α and proliferation and also inhibits binding of HIV-1 to the CD4 receptor. The art teaches that T cell activation is important to initiate and maintain inflammatory process, and to upregulate membrane receptors. The art also teaches that T cell activation enhances entry and replication of different viruses including that of HIV-1 entry and replication (see Jenkins M K, Annual Review of Immunol, 2001, vol 19, p 23-45. For HIV-1 Huber B T, Microbiological Reviews, 1996 vol 60 p 473-82 for EBV, CMV Rabies virus; Sutkowski N, Immunity, 2001, vol 15, p 579-89 for EBV; Frenkel N, J of Virol, 1990, vol 64 p 4598-602 for Herpes Virus 6; and Stein B S, Advances in Exp Med and Biol, 1991, vol 300 p 71-86 for HIV-1). Accordingly IgM-ALA by inhibiting T cell activation has an inhibitory effect on inflammatory processes in different disease states and at different tissue sites as well as has an inhibitory effect on replication of HIV-1 virus and other viruses which are dependent on activation of T-Cells and other cells for viral replication.

The art also teaches that HIV-1 virus attaches to the CD4 receptor and enters cells through binding of the virus to chemokine receptors (e.g. CXCR4 and CCR5), which internalizes after viral binding. The art also teaches that replication of HIV-1 within the cell is enhanced with T cell activation. The art also teaches that IgG anti-CD4 antibodies inhibit HIV-1 viral entry and that T cells can be rendered inactive by IgG antibodies that target CD3 or CD4. Hence the inventor believes that IgM-ALA inhibit HIV-1 infection (i) by inhibiting HIV-1 virus binding to CD4 and chemokine receptors, (ii) inhibiting HIV-1 induced internalization of chemokine receptor and (iii) by inhibiting T cell activation, thus inhibiting viral replication.

The art also teaches that certain viruses bind to non-chemokine receptors on lymphocytes. Polio virus binds to CD15 receptor, Hepatitis C virus binds to CD81 and other lymphocyte membrane receptors. Herpes virus 6 bind to a T lymphocyte receptor that has not been identified while the EBV virus binds to the C21 receptor on B lymphocytes. Measles virus binds to receptors present on B and T lymphocytes. (See Dimitrov D S, Nature Reviews (Microbilogy), vol 2 p 109-121, 2004 for polio and other viruses; Barel M, Eur J of Immunol, vol 33, p 2557-2566, 2003 for EBV virus; Frenkel N, J of Virol, vol 63, p 4598-4602, 1990 for Herpesvirus 6 and Chong T W et. al., J of Surgical Research, Vol. 130, p. 52-57, 2006). The art also teaches that replication of these viruses is enhanced with activation of these cells. Hence the inventor believes that these heterogenous and polyreactive IgM-ALA will inhibit infectivity of these viruses by binding to non-chemokine receptors involved in viral entry and cell activation.

The art also teaches that many inflammatory processes are initiated by T cell activation, with enhancement of cytokine production and chemotaxis of cells. The art also teaches that IgG antibodies that inhibit T cell activation (e.g. anti-CD3 or anti-CD4), chemotaxis (e.g. anti-RANTES or anti-CCR5), and cytokines especially anti-TNF- will also inhibit inflammatory processes e.g. acute allograft rejection, bowel inflammation and rheumatoid arthritis. Accordingly, the inventor believes that IgM-ALA inhibits inflammatory processes, by inhibiting T cell activation, proliferation and cytokine production and by inhibiting chemotaxis of inflammatory cells.

The inventor will now briefly provide a summary of chemokines and chemokine receptors. Details on this subject are described by Olson and Ley, Amer. J Physiol Regulatory Integrative Comp Physiology 283: R7-R28, 2002; by Gerard and Rollins, Nature Immunol 2: 108-115, 2001; and by Onuffer and Horuk, Trends in Pharmacological Sciences 23: 459-467, 2002.

The known chemokine system in humans comprises, approximately 50 different chemokines and about 20 G-protein coupled chemokine receptors. The chemokine system has several characteristics (i) Most chemokines are secreted but some e.g. fractalkine are expressed on the cell surface. (ii) Chemokines are subdivided into CC, CXC, or CX₃C groups based on the number of amino acids between the first two cysteines (iii) Certain chemokines bind only one receptor e.g. CXCR4 with SDF-1α and CXCR5 with BCA-1 while other receptors can bind to several chemokines e.g. CXCR3 binds to IP-10, Mig and I-TAC. Similarly, a single chemokine can bind to several receptors e.g. RANTES will bind to CCR1, CCR3 and CCR5 with high affinity. This has led many in the field to suggest that the chemokine system was rife with redundancy. However, there are certain exceptions as lack of CXCR4 receptor expression is associated with abnormal embryogenesis and organogenesis. In addition, different chemokine receptors expressed on the same cell can induce specific signals, thus indicating that receptors are coupled to distinct intracellular pathways. (iv) Certain chemokines (and their respective receptors), important for normal homeostatic trafficking (e.g. BCA-1, which is involved with normal migration of lymphocytes to lymph nodes), are constitutively expressed while inflammatory chemokines (and their receptors) are induced on leucocytes and other cells e.g. endothelial cells, only under specific conditions, typically by inflammatory chemokines e.g. IL-1 or TNF-α produced by macrophages or activated T lymphocytes. (v.) Chemokine receptors are expressed on many different cells including leucocytes, endothelial cells, smooth muscle cells, and epithelial cells and neuronal cells and these cells can also secrete chemokines.

Chemokines play a prominent role in leucocyte and fibrocyte trafficking that occurs with several inflammatory processes as diverse as multiple sclerosis, rheumatoid arthritis, systemic lupus erythematosus, vasculitis, allograft and xenograft rejections, acute and chronic bacterial and viral infections, asthma, colitis, inflammatory bowel disease, interstitial lung inflammation and fibrosis, psoriasis, atherosclerosis, hypertension, ischaemia-reperfusion and inflammation associated with neoplasia and hepatitis. All these diseases will be referred to as “inflammatory states”. Additionally, chemokines play a role in other non-inflammatory processes e.g. organo-genesis, hematopoiesis, and neuronal communication with microglia and with angiogenesis. The pivotal role played by chemokines in some of these disorders is illustrated by the observation that (a) specific deficiency of CXCR4 is associated with abnormal organo-genesis and (b) individuals with a homozygous defect in CCR5 are protected from allograft rejections and asthma. The participation of the chemokine system in inflammatory states involves trafficking of fibrocytes and leucocytes as well as leucocyte activation and immune cell differentiation. For example, chemokines induce neutrophils to increase integrin expression, neutrophil degranulation and super oxide formation. Similarly, the chemokine system is involved in tissue-specific homing of lymphocyte subsets to lymphoid organs where lymphocytes get activated and start differentiating (see Olson and Ley reference).

Of particular significance is the finding that CD4 and chemokine receptors i.e. predominantly CXCR4 and CCR5 act as co-receptors for the entry of HIV-1 virus into cells. The X4 HIV-1 virus uses the CXCR4 receptor while the R5 HIV-1 virus uses the CCR5 receptor. It has become abundantly clear that viral entry through binding of HIV-1 to CD4 and chemokine receptors is of prime importance in influencing viral replication and disease progression after an HIV-1 infection. For example, individuals with genetic defects in the CCR5 receptor have been associated with a prolonged latency period after HIV-1 infection i.e. a slower progression of HIV-1 to AIDS.

Researchers and pharmaceutical companies have been looking into strategies to block or inactivate CD3, CD4 and specific chemokine receptors in an effort to inhibit T cell mediated inflammatory processes that induce disease processes and to inhibit HIV-1 entry into cells. With regard to HIV-1, some strategies include use of peptides and IgG monoclonal antibodies (produced after immunization) that will bind to specific chemokine receptors. Other strategies include use of IgG antibodies or soluble receptors (e.g. soluble CD4) that bind to the HIV-1 virus envelope to prevent attachment of HIV virus to leucocyte coreceptors. Choudhory et. al. teaches (See Expert Opinion Biol. Ther. 2006, Vol. 6, p. 523-531) that the use of IgG antibodies, specific for viral co-receptors on leucocytes (e.g. anti-CD4 or anti-CCR5), has had limited success as such antibodies interfere with the receptor immunological function (e.g. anti-CD4, anti-CXCR4) or has led to cell depletion as such IgG antibodies after binding to cell membrane receptors, are cytolytic at body temperature (See Lobo P I et al, Transplantation, Vol. 26, p. 84-86, 1978). IgG induced cytolysis is mediated by complement activation or antibody dependent cellular cytotoxicity (ADCC). Furthermore antibodies that bind to a few receptor epitopes (e.g. anti-CCR5 (2D7)) can only inhibit certain R5 isolates of HIV-1. Similarly IgG anti-HIV-1 antibodies or soluble CD4 has had limited success primarily because the HIV-1 viral envelope constantly mutates and secondly because serum inhibits the function of these IgG anti-viral monoclonal antibodies and soluble CD4 (See Choudhry V. et. al., Expert Opin. Biol. Ther. 2006, Vol. 6, p. 523-531 for a review of this prior art). Strategies to inhibit inflammatory processes e.g. acute allograft rejection, rheumatoid arthritis, include use of monoclonal or polyclonal IgG antibodies that bind to T cell receptors (e.g. CD3, CD4), chemokine receptors or certain pro-inflammatory cytokines e.g. antibodies to TNF-. Such strategies suffer from the same problems as indicated for strategies to inhibit HIV-1 and in addition require maintenance immunosuppression as the IgG antibody effect is short lived. IgM-ALA has several advantages when compared to these strategies in prior art. IgM-ALA does not lead to leucocyte depletion as IgM-ALA are not cytolytic at body temperature and the inhibitory effects of IgM-ALA on inflammation and HIV-1 are not inhibited by serum. Additionally IgM-ALA specifically inhibits production of TNF-, which has a major role in inflammatory disease (See McDevitt, H et. al., Arthritis Res. Vol. 4, (Supp 31): S141-S152, 2002). Finally, maintaining high levels of IgM-ALA (e.g. with a vaccine approach) has the added benefit of requiring no or minimal maintenance immunosuppression. Hence the inventor believes that the current invention of administering IgM-ALA to treat diseases or enhancing production of in-vivo IgM-ALA is novel over prior art.

Finally, the inventor will provide a summary on the role of T cells in inflammatory processes. Prior art has shown that T cells play a prominent role in several diverse inflammatory processes including allergy, autoimmune disorders, rejection of transplant organs, atherosclerosis, and resistance to infections. For example, allografts are not rejected in T cell deficient animals indicating that T cell activation and cytokine production is necessary to induce or facilitate the inflammatory process associated with rejection. The art also teaches that CD3, CD4, and CD86 are important receptors (or switches) that are involved in T cell activation (see Werlen G, Current Opinion in Immunol Vol 14 p 299-305, 2002 for prior art in this regard). The inventor therefore believes that IgM-ALA NAA by binding to CD3 and CD4 will inhibit T cell activation and provide another mechanism to inhibit diverse inflammatory processes where T cells activation plays a prominent role. Examples on the role of T cells in some inflammatory processes are reviewed in Perkins D L, Current Opinion in Nephrology and Hypertension Vol 7, p 297-303, 1998; Hansson G K et al Circulation Research Vol 91 p 281-291, 2002. There is prior art to show that cytokines and chemokines are involved in the inflammatory process. Certain cytokines and chemokines are pro-inflammatory while others are anti-inflammatory. Prior art has shown that TNF- in particular, is the major cytokine that enhances inflammation in rheumatoid arthritis, psoarsis and Crohn's disease. Inhibitors of TNF-have a marked beneficial effect on these particular inflammatory disorders (see Feldman M, Annual Rev Immunol 2001, vol 10, p 163-196; Sandborn W J, Inflamm Bowel Dis 1999, vol 5, p 119-133; and Chaudhari U, Lancet 2001, vol 357 p 1842-1847). In the present application, inventor has demonstrated that IgM-ALA inhibits leucocyte secretion of TNF- and other chemokines. Inventor believes that inhibition of chemokines and cytokines by IgM-ALA could provide another mechanism for inhibiting an inflammatory process.

Researchers and pharmaceutical companies have been looking into strategies to inhibit T cell activation in an effort to inhibit inflammatory processes including autoimmune disorders, allergies and allograft rejections. Some of these include use of antibodies that inactivate or kill T cells. These antibodies are produced by immunizing animals with human T lymphocytes. Other strategies include use of (i) immunosuppressive drugs e.g. cyclosporine or Rapamycin and (ii) agents that inhibit cytokines (e.g. TNF-) that are produced by activated T cells. Such strategies are expensive and have serious side effects and have to be taken for prolonged periods and at times for life especially after a transplant. Strategies to inhibit T cell mediated inflammatory responses by administering IgM-ALA and enhancing production of IgM-ALA in vivo may prove to be much less expensive, more effective and available for large populations of individuals.

### SUMMARY OF THE INVENTION

In the present application, the inventor claims that IgM anti-lymphocyte auto-antibodies (IgM-ALA) can be used to treat infective and inflammatory states and the inventor believes that this invention is novel over prior art for the following reasons. Firstly, the inventor shows that IgM-ALA are naturally occurring autoantibodies (NAA) that will bind to lymphocyte receptors important in T cell activation, leucocyte chemotaxis and receptors that are important for viral entry into cells especially entry of HIV-1 and Hepatitis C viruses. These IgM-ALA are not cytolytic to normal cells at 37 C and hence by binding to lymphocyte receptors at body temperature these IgM-ALA are best suited to modulating lymphocyte function, chemotaxis and acting as “blocking antibodies” to inhibit viral entry into cells. Secondly, the inventor shows that IgM-ALA are effective in in-vivo models of rejection, HIV-1 infection and in the presence of human sera. Thirdly, IgM-ALA by binding to lymphocyte receptors inhibit the production of certain pro-inflammatory cytokines especially TNF-, which has a major role in inflammation. IgM-ALA are distinct from prior art that use IgG antibodies which bind to lymphocyte receptors as such IgG antibodies are not naturally occurring, are cytolytic to cells at 37 C, bind to few epitopes on the receptors (and hence cannot effectively block multiple viral isolates) and their effect on receptors is inhibited by normal sera (probably by anti-idiotypic antibodies in sera). Hence, unlike IgM-ALA, IgG-ALA are not very effective in-vivo as their effect is short lived (secondary to inhibitors in serum) and these IgG antibodies cause lymphopenia. The IgG anti-CD4 antibody present in IVIG preparations has not been shown to be naturally occurring and IVIG has not been shown to be effective in-vivo to treat HIV. Inventor has shown that the inhibitory effect of IVIG on HIV-1 is inhibited in presence of sera. Inventor has also shown that inhibitory effects of IgM is mediated by significantly less (approximately 5 to 10 fold less) IgM when compared to pooled normal or HIV IgG.

IgM NAA are mostly polyreactive in that a single IgM monoclonal antibody can recognize several closely similar self-antigens, which possess a unique but distinct set of epitope specificities. Hence, a monoclonal IgM that binds to one receptor will very often bind to similar receptors belonging to the same class, e.g. an IgM antibody to CCR5 could bind to another chemokine receptor e.g. CCR1 and also bind to chemokine receptors expressed in animals. Conversely animal IgM NAA will bind to similar cell receptors as human IgM NAA.

While the presence of IgM anti-lymphocyte NAA or IgM-ALA has previously been described, there is no prior art identifying the lymphocyte receptors targeted by IgM, nor is there prior art showing that IgM-ALA can inhibit T cell function or inhibit viral infectivity of cells, or inhibit cytokine production or inhibit chemotaxis.

In the present invention, applicant has discovered that some of the IgM obtained from normal human sera and umbilical cord blood bind to CD4, CD81 and chemokine receptors and specifically inhibit binding of chemokines to their receptors, inhibit chemotaxis and inhibit HIV-1 and hepatitis C virus from infecting cells. The inventor has also shown that IgM inhibit T cell activation, inhibit cytokine production and inhibit T cell proliferation. Absorption of IgM with leucocytes removes the inhibitory effect of IgM on chemotaxis, T cell proliferation and on HIV-1 infectivity. Accordingly, the inventor believes that IgM-ALA in the IgM preparation inhibits HIV-1 infectivity by “blocking” HIV-1 entry through binding to CD4 and the chemokine receptor as well as by inhibiting lymphocyte activation. The inventor has also discovered that IgM-ALA inhibits Hepatitis C from infecting lymphocytes and inhibition is mediated by inhibiting Hepatitis C viral entry as IgM-ALA binds to CD81 and other lymphocytes receptors important for hepatitis C viral entry. Absorption of IgM with leucocytes removed the inhibitory effect of IgM on Hepatitis C infectivity of cells.

Moreover, IgM-ALA are a heterogeneous group of several different antibodies that bind to chemokine and other non-chemokine receptors on the lymphocyte. Such non-chemokine receptors include glycoprotein, glycolipid and phospholipid receptors. These IgM anti-lymphocyte NAA have low binding affinity and do not lyse normal cells in the presence of complement at body temperature (i.e. 37° C.). Applicant, in this invention has discovered that these polyreactive IgM-ALA bind to the same or closely similar lymphocyte receptors that are also present on other leucocytes (i.e. neutrophils, eosinophils, and macrophages), endothelial cells, hepatocytes and malignant cells (both lymphoid and non-lymphoid). In the present invention, applicant also demonstrates that IgM-ALA binds to a non-chemokine receptor, identified as CD3 and CD4 and further shows that naturally occurring IgM with anti-CD3, anti-CD4 and anti-chemokine receptor activity inhibits lymphocyte activation and proliferation. Applicant also demonstrates that IgM-ALA antilymphocyte NAA downregulates CD2 and CD4 on T cells and CD80, CD86 on macrophages, (which are antigen presenting cells) thus inhibiting T cell activation through this additional mechanism.

Prior art has shown that natural autoantibodies (NAA) are predominantly of the IgM isotype with some having the IgG isotype. NAA of the IgG isotype has been shown in prior art to have reactivity to serum proteins (e.g. Factor VII, C3b complement), intracellular proteins (e.g. myosin), nucleo-proteins (e.g. DNA), phospholipid extracts (e.g. cardiolipin) and red blood cell antigens. The present invention is distinct from prior art of IgM-NAA in that one cannot demonstrate IgG-antilymphocyte autoantibodies (IgG-ALA) even though the cell membrane has phospholipids. The inventor believes that B-1 lymphocytes fails to produce IgG-ALA under physiological conditions as IgG antibodies can cause cytolysis at 37 C and will lead to lymphocyte depletion. Prior art teaches that lack of red blood cell (RBC) cytolysis, despite naturally occurring IgG-anti RBC, can be explained on basis of IgM-NAA that bind to the variable region of IgG-anti RBC thus inhibiting binding of IgG to RBC under normal conditions. (See Stahl D., et. al. Current Pharmaceutical Design, 2003, Vol. 9, p. 1871-1880). There is no prior art to show presence of IgG-ALA under physiological conditions in umbilical cord sera and in normal sera. Prior art however shows presence of IgG-anti CD4 in disease sera (e.g. SLE) or in pooled IVIG. The inventor believes that presence of IgG anti-CD4 in SLE or in pooled IVIG preparations results from immune dysregulation (e.g. SLE) or produced by non-B1 cells in response to cross-reactive non-self antigens (e.g. in IVIG).

The inventor has observed that human kidney transplant recipients, who have high levels of IgM reactive to their donor lymphocytes, rarely have problems with rejections. The inventor also shows in this application that IgM knockout mice have a more severe and accelerated rejection after a heart transplant. Applicant, in this invention, believes that protection against rejection is mediated by the inhibitory effect of IgM on autologous leucocytes, including T cells and donor endothelial cells. High level binding of recipient IgM to donor lymphocytes is also associated with similar level of IgM binding to recipient leucocytes and donor endothelial cells. Recipient IgM would thus protect against rejection by inhibiting leucocyte chemotaxis as well as by inhibiting activation of autologous lymphocytes e.g. through binding to CD3 and CD4 and chemokine receptors.

The inventor has observed increased apoptosis of malignant cells, (but not normal cells) at 37° C. in presence of normal IgM anti-lymphocyte NAA. The inventor believes that these antibodies also protect against malignancy by enhancing apoptosis and also by inhibiting metastatic spread of malignant cells, mediated through chemokine receptors. There is prior art to show that metastatic spread of malignant cells and growth of malignant cells is enhanced by chemokine receptors.

Prior art has shown that B-1 lymphocytes which produce IgM-NAA (and other NAA isotypes) are distinct from B-2 lymphocytes that produce antibodies in response to non-self antigens. B-1 lymphocytes, unlike B-2 lymphocytes, do not produce antibodies (with specificity for non-self antigens when stimulated with non-self antigens (in absence of adjuvants). B-1 cells are stimulated with self antigens. How then do infective or inflammatory states enhance production of NAA (including IgM-ALA)? Prior art (See Baumgarth N., et. al., Springer Semin Immuno, 2005, Vol. 26, p. 347-362) shows that B-1 cells are stimulated to produce NAA in response to mitogens and cytokines. The inventor therefore believes that mitogenic antigens produced in infective/inflammatory states could exert a mitogenic effect on B-1 cells. Furthermore the release of cytokines in infective/inflammatory states could stimulate B-1 cells to enhance NAA production. The third objective is to provide a method for producing IgM-ALA in-vitro and in-vivo.

Accordingly, one object of the present invention is to provide methods of inhibiting disease processes involving and/or mediated by chemokine and non-chemokine receptors through use of IgM anti-lymphocyte NAA (IgM-ALA) or enhanced in-vivo production of IgM-ALA. Another objective is to provide a method for quantitating levels of IgM-ALA with different receptor specificities to aid in preventing disease progression.

The above and other objects, advantages and features of the present invention will become more apparent from the following detailed description of the presently preferred embodiments, when considered in conjunction with the figures, and to the appended claims.

### DISCLOSURE OF INVENTION

To achieve the foregoing and other objects, and in accordance with the purpose of the present invention as embodied and broadly described herein, the present invention relates to the expression, stimulation and administration of IgM receptor-binding antibodies to address viral infections and disease states induced thereby.

Prior art has shown that IgM autoantibodies present in the blood of normal uninfected individuals and in newborns bind to extracellular receptors present on lymphocytes. There is prior art to also show that IgM autoantibodies to lymphocytes, which are present at low levels in normals, increase in various infectious states (including HIV and Hepatitis C), autoimmune disorders, and inflammatory disorders. These IgM antibodies are heterogenous and bind to several different membrane receptors including glycoprotein, glycolipid, glycosphingolipid and phospholipid membrane antigens on the lymphocyte membrane. These IgM autoantibodies do not damage normal cells at 37° C. as at that temperature they have a low binding affinity, cannot activate complement or ADCC and therefore at 37 C operate as “blocking” antibodies or are there to alter cell function.

According to the present invention, IgM anti-lymphocyte auto antibodies present in normal sera bind to chemokine receptors, for example, CXCR4, CCR5, CCR3 and CCR2b and other non-chemokine lymphocyte-surface receptors e.g. the CD3, CD4 and CD81 antigen. The inventor also shows that IgM anti-lymphocyte antibody inhibits HIV-1 and hepatitis C from infecting cells.

While not wishing to be bound to a specific theory, the inventor believes that the increase in these antibodies after an HIV-1 infection, slows down the progression of the infection towards development of AIDS and the high levels of IgM in newborns protect newborns from getting HIV-1 viremia from their infected mothers. Only 20 to 25% of babies, born of untreated mothers infected with HIV-1, are found to have the HIV-1 virus. Mechanisms for inhibiting HIV-1 infectivity of cells include, (but are not limited to): (i) inhibiting binding of HIV-1 to the CD4 receptor (ii) “blocking” of HIV-1 viral entry through binding of IgM to chemokine receptors (iii) inactivation of lymphocytes by binding to the CD3 and CD4 receptor or downregulating other activating receptors e.g. CD2, CD4, CD80, CD86 and chemokine receptors and inhibiting internalization of chemokine receptors after HIV-1 binds to these receptors. Lipid rafts contain glycosphingolipids as well as phospholipids, which prior art has shown to be target antigens for IgM anti-lymphocyte autoantibodies. The binding of IgM anti-lymphocyte NAA to glycolipids and phospholipids has been described in Griggi et al, Scand. J of Immunol, 40: 77-82, 1994 and Stimmler et al, Archives of Internal Medicine 149: 1833-1835, 1989.

The inventor also believes that the increase in IgM-ALA after Hepatitis B or C and malaria slows down the progression of the infection by binding to cell membrane receptors used by these infective agents for cell entry. Examples of cell receptors include (but are not limited to) CD81, low density lipoprotein receptor (LDL), human scavenger receptor B-1, and glycosaminoglycan receptor. These receptors are present on lymphocytes and hepatocytes and are used by the hepatitis virus and the malaria parasite for entry into hepatocytes and other cells. Both malaria and the hepatitis virus replicate in these cells once they gain entry.

Chemokines, chemokine receptors, and other lymphocyte receptors (e.g. CD3, CD4 and other co-stimulatory molecules) are involved in inflammatory processes that involve leucocytes and endothelial cells. Examples of inflammatory processes include (but are not limited) auto-immune disorders (e.g. SLE, rheumatoid arthritis), asthma, atherogenesis, end-stage renal disease (ESRD) patients on dialysis, sarcoidosis, various viral, bacterial and parasitic infections, allograft and xenograft rejections, various forms of vasculitis, multiple sclerosis, interstitial lung and kidney inflammation and glomerulonephritis. While not being bound to a specific theory, the inventor believes that IgM anti-lymphocyte NAA through binding to chemokine receptors and other lymphocyte receptors could inhibit the above-mentioned inflammatory processes. Potential mechanisms for inhibition would include inhibition of chemokine receptor function after binding of IgM and more importantly inactivation of lymphocytes and/or macrophages after binding to chemokine receptors and non-chemokine receptors as for example, the CD3, CD2, CD4 and CD86 receptor.

IgM anti-lymphocyte NAA also bind to endothelial cells and malignant cell lines. In this invention we show that IgM NAA, are polyreactive and hence, bind to the same or closely similar receptors present on these cells. The inventor believes that some monoclonal IgM anti-chemokine receptor antibodies are polyreactive and bind to several different chemokine receptors as absorption of IgM with lymphocytes removes the IgM that binds to malignant cells, Neutrophils, eosinophils, macrophages, and endothelial cells even though these cells have different chemokine receptors and lack chemokine receptors present on lymphocytes.

It is believed that IgM by binding to chemokine and other non-chemokine receptors on endothelial and malignant cells inhibit the function of these cells. For example, there is prior art to show that chemokine receptors on malignant cells contribute to metastases of these cells (see Mueller A et al, Nature Vol 410 p 50-56, 2001 and Gerard C, Nature Immunol Vol 2 p 108-115, 2001). The inventor therefore believes that IgM, by binding to chemokine receptors on malignant cells and/or endothelial cells could inhibit the growth and spread of malignant cells. Furthermore there is prior art to show that lymphocytes in lymph nodes and infiltrating leucocytes within the tumor mass secrete chemokines and other cytokines, all of which contribute to growth and metastases of tumor cells. The inventor therefore believes that IgM by binding to chemokine receptor and other “activation” receptors on leucocytes and malignant cells will, through this additional mechanism, also inhibit tumor growth and metastases. Finally, the inventor shows that malignant lymphoma cells (but not normal cells) have enhanced cell death at 37° C. when incubated with IgM. Hence, IgM through enhancing cell death of malignant cells could provide yet another mechanism for an anti-cancer effect.

Endothelial cells and leucocytes are also important in several inflammatory processes (e.g. allograft rejections, atherogenesis, vasculitis and inflammatory states of the brain). It is therefore believed that IgM anti-lymphocyte NAA by binding to chemokine receptors on leucocytes and endothelial cells could provide a protective role in inhibiting such inflammatory processes. Furthermore, IgM anti-lymphocyte NAA could also inhibit inflammatory processes by inhibiting receptors (e.g. CD3 and CD4) that activate T lymphocytes and macrophages as well as by decreasing production of certain pro-inflammatory cytokines especially TNF-.

The inventor believes that pooled or polyclonal IgM preparations contain a heterogenous group of antibodies that bind to chemokine or non-chemokine receptors on leucocytes, endothelial cells and malignant cells and that the binding of IgM to several of these receptors may add or have a synergistic effect in IgM mediated inhibition of viral infectivity, inflammatory states and malignant cell growth and spread.

Experimental Studies

Methods/Procedures

Cell Lines

Sup T-1 and Jurkat are human lymphoma T cell lines constitutively expressing the CXCR4 receptors. U937 is a human monocytoid cell line expressing CD4, CXCR4, CCR5 and other chemokine receptors e.g., CCR2b. These cell lines are obtained from the AIDS Reagent Program or ATCC at NIH.

An HOS osteosarcoma cell line is co-transfected with CD4 and either CXCR4 or CCR5 or CCR3 or CCR1 genes to produce HOS-CD4, HOS-CD4-CXCR4 and HOS-CD4-CCR5HOS-CD4-CCR3 and HOS-CD4-CCR1 cells. GHOST CCR5 and GHOST CXCR4 are HOS-CD4 cells co-transfected with the HIV-2 LTR driving hGPP construct and either CCR5 or CXCR4 genes, respectively. The cell line and the transfectants are obtained from the AIDS Reagent Program at NIH.

A glioblastoma cell line, U373-MAGI, is co-transfected with CD4 and either CXCR4 or CCR5 to produce U373-MAGI-CXCR4 and U373-MAGI-CCR5, respectively. Again, the cell line and the transfectants are obtained from the AIDS Reagent Program at NIH.

All of the transfected cell lines stably express CCR5 or CXCR4, with the U373-MAGI cells having the highest expression of these receptors.

Human peripheral blood lymphocytes (“PBL”) is activated with phytohemagglutinin and IL-2 to enhance CCR5 and CXCR4 expression. PBL (2×10⁶ cells in 1 ml RPMI culture media containing 10% fetal calf serum are activated by initially pre-treating Ficol/hypaque separated PBL with IL-2 (40 units/ml) and phytohemagglutinin (“PHA-P”, 5 mcg/1 ml) and then washing the PBL after the cells are cultured at 37° C. in about 5% CO₂ for 24 to 48 hours. Such PHA pre-treated cells are then kept growing for about another 6 to 7 days supplemented with 20% fetal calf serum and IL-2 (40 units/ml) before being used in chemokine binding assays.

HIV-1 Viruses

The R5HIV-1 viruses (8397, 8442, and 8658) used to infect GHOST CCR5 or mitogen activated PBL are obtained from Dr. Homayoon Garadegan at Johns Hopkins University. The X4 virus IIIB and RF used to infect GHOST CXCR4 or mitogen activated PBL is obtained from the AIDS Reagent Program at NIH.

IgM Preparations and Sera

Studies were performed with IgM that was purified from heat-inactivated sera (56° C.) of normal individuals or from patients. IgM was prepared from sera with Sephacryl S-300 HR size exclusion column chromatography. IgM was not precipitated from sera with hypotonic dialysis or by ammonium chloride precipitation as these processes reduced the biological activity. Any contaminating IgG was removed from the IgM preparation by re-passage of purified IgM through a Sephacryl/S-300 HR column and by exposure to Agarose-protein G and Agarose bound to goat anti-human IgG (Sigma). We employed size column chromatography basically to remove low molecular weight substances (e.g. chemokines, anti-viral drugs) and IgG anti-HIV-1 antibodies that could affect our data. Serum protein electrophoresis and immunoelectrophoresis revealed that these IgM preparations, obtained by size exclusion chromatography, contained IgG (<1%), albumin (<3%), and other proteins (<1%). We did not want to affinity purify these antibodies as such procedures, e.g. binding of IgM to mannan binding protein or binding of IgM to agarose coupled with goat anti-human IgM yielded 10-15% of the starting IgM and has the potential of depleting certain IgM subsets. Instead, in several experiments we used IgG, IgA, albumin and alpha 2 macroglobulins to determine if our observations could be explained by some of these minute contaminants. No detectable RANTES and SDF-1α was present in these IgM preparations when analyzed by ELISA and Western blot techniques.

We obtained sera from normal uninfected healthy individuals, asymptomatic patients with HIV-1 infection, and on HAART therapy, patients with end stage renal disease (ESRD) on dialysis and patients with hepatitis C infection. Some of the HIV-1 patients had suffered AIDS defining illnesses and some other had high viral loads (>100,000 copies) despite HAART therapy. To obtain a sufficient quantity, IgM from nine HIV-1 patients was pooled. IgM from seven ESRD patients was also pooled. Data presented in figures are either from individual or from pooled IgM and are indicated in the figures.

The culture supernatants of EBV transformed human B cell clones are separated by Sephacryl S-300 HR column chromatography, which separates proteins by size. The human B cell clones are derived from B lymphocytes isolated from the blood of a patient with SLE and from the umbilical cord of a healthy newborn baby. The B cell clones are developed by infecting B cells with the EBV virus, which makes the B cells immortal and capable of secreting antibody, i.e., IgM. More particularly, non-T cells are isolated from PBL after removal of T cells using a magnetic bead kit obtained from Dynal Biotech, Oslo, Norway. About 2×10³ non-T cells in about 0.1 ml RPMI 1640 cell culture media containing about 10% fetal calf serum are added to each well of a 96 well plate. To each well is then added about 50 lambda of EBV-containing B95-8 cell line supernatant. Before incubation, about 10⁴ allogenic irradiated (3,000 rads) PBL in 0.05 ml are added as feeder cells. The plates are incubated at 37° C. in about 5% CO₂. The culture medium is replaced about every 4 to 5 days. After about 3 to 4 weeks, B cell lines appear as “clumps” in the wells. Feeder cells die during this period. When the “clumps” appear, these clumped cells are transferred to a 24-well plate, i.e., cells from one well are transferred into a single larger well. Culture media is changed when the media changes to a yellowish color, usually about 3 to 5 days. After about 2 weeks, supernatants are checked for IgM antibody. Wells containing lines with desired antibody specificity are further subcloned with limiting dilution in a 96-well plate. About 10⁵ feeder cells are added to each well containing these lines. Supernatants are rechecked to isolate clones with desired antibody specificity. Supernatants are refrigerated, but not frozen as IgM can precipitate out. Clones secreting IgM-ALA are cryopreserved. Supernatants from such clones usually contain about 0.5 to about 0.7 μg/ml antibody. Clones of particular interest can be fused with K6H6/B5 plasmacytoma cell line (or other similar cell lines that do not secrete antibodies) to develop hybridomas. The clones are screened to identify and obtain those clones that react with CD3, CD4, CCR5 and CXCR4 chemokine receptors present on the transfected cells. Such clones have increased IgM binding by flow cytometry to the HOS-CD4 transfectants (i.e., HOS-CD4-CXCR4 and HOS-CD4-CCR5) when compared to the HOS-CD4 control. Two clones, CK12 and CK15 secreting IgM with increased binding to HOS-CD4 CCR5 or CXCR4 transfectants were identified in this manner and obtained from B cells of the SLE patient. CK12 only bound to HOS-CD4-CXCR4 while CK15 was polyreactive and bound to HOS-CD4-CCR5, HOS-CD4-CCR3 and HOS-CD4-CCR1. Clones from umbilical cord B cells include 2E11-1H4, 4D1, 4G4, 1E12-G3, all of which secreted IgM that bound to membrane receptors on lymphocytes (IgM-ALA) except for 2E11-H4 that secreted IgM without IgM-ALA.

Any contaminating IgG is removed from the IgM preparations that are isolated from the sera and the culture supernatants by exposure to both protein G-Agarose (available from Sigma) and goat anti-human IgG (Fc specific)-Agarose (available from Sigma).

IgM is also obtained using Sephacryl S-300 HR column chromoatography from sera of a patient diagnosed with Waldenstrom macroglobulinemia (a form of B cell lymphoma) and which, on serum protein electrophoresis, has a single peak for IgM (monoclonal). This latter IgM preparation is hereinafter referred to as “Waldenstrom IgM” and the monoclonal IgM binds to an undefined membrane receptor on lymphocytes and other leukocytes

IgM was also obtained from pooled sera of mice, rats, goats and rabbits. We used similar techniques as for human sera to obtain purified animal IgM.

IgG was also obtained from normal, HIV-1 and ESRD sera using the same techniques as for separating IgM i.e. using Sephacryl S-300HR column chromatography except the second protein peak from the column eluates was used to isolate IgG.

Absorption of IgM with Jurkat and U937 Cells

2.5 ml IgM at 0.2 mg/ml in RPM1 was absorbed for 45 minutes with 280×10⁶ Jurkat cells and 200×10⁶ U397 cells at 37° C. in 5% CO₂. We used Jurkat and U937 cells as these cells express most of the leucocyte membrane receptors including CD3, CD4 and chemokine receptors. The IgM was centrifuged at the end of 45 min to remove cells and the absorbed IgM was quantitated using ELISA techniques. 25 to 30 percent of IgM was lost in the absorption technique. Absorbed IgM had <5% residual binding activity to Jurkat cells, U937 cells, lymphocytes, neutrophils or cultured endothelial cells as determined by flow cytometry.

Absorbing Out IgM Anti-CD4 from Purified IgM

200 μg purified IgM in PBS containing Ca++ and 0.1% BSA was absorbed with recombinant CD4, immobilized in a 96 well immulon plate where each well was coated with 500 ng recombinant soluble CD4 using the Immuno-Tek ELISA construction kit (Zepto Matrix, N.Y.). Each absorption required 0.5 μg IgM to be incubated with 500 ng immobilized CD4 for 45 min at RT.

Preparation of Monomeric IgM

Monomeric IgM was made from the pentameric form in 200 nM Tris, 150 mM NaCl, and 1 mM EDTA, pH 8.0, and by reduction with 5 mM DTT for 2 hour at room temp. Subsequent alkalinization was performed for 1 hour on ice with 12 mM iodoacetamide. IgM monomers were isolated from any remaining pentameric forms by column chromatography (Superdex-200) equilibrated with PBS. Purity of monomeric IgM was confirmed with SDS-PAGE Western blots under reducing and non-reducing conditions. With flow cytometry, one observed less than 20 percent reduction in binding of monomeric IgM to lymphocytes when compared to the pentameric form.

Chemokines

RANTES, SDF-1α and biotin-labeled SDF-1α-MIP-1α and RANTES are obtained from Becton Dickinson of La Jolla, Calif. Radio-labeled RANTES (referred to as “¹¹²⁵ RANTES” or “I¹²⁵”) is obtained from NEN Life Science of Boston, Mass. RANTES binds to CCR5, while SDF-1α binds to CXCR4.

Antibodies

Clones 2D7, CTC-5, 45502, 45523, and 45549 are murine IgG monoclonal antibodies specific for CCR5 when expressed on intact cells. Clone CTC-5 in addition binds to linearized CCR5 in Western blots. Clones 12G5 (IgG 2a) 44708, (IgG 2a) 44717 (IgG 2b), and 44716 (IgG 2b) are murine IgG monoclonals that bind to CXCR4 receptors on intact cells and neutralize chemotaxis in response to SDF-1α. All these antibodies were obtained from R&D Systems or the NIH AIDS Reagent program. Clone 4G10, a murine IgG monoclonal that binds to the N-terminal region of CXCR4 was a kind gift from Dr. Chris Broder. The following IgG anti-CD4 murine monoclonals were used Leu3a (Becton-Dickenson), Sim2 and Q4120 (AIDS Reagent Program at NIH) and MT310 (Dako).

IgM Inhibition of Chemokine Binding to Receptors on Intact Cells

Normal, ESRD and HIV IgM have a similar inhibitory effect on binding of biotin labeled SDF-1α and MIP-1α to cells. Cells (0.5×10⁶ in 0.5 mL) obtained from T cell lines (Hut 78 and Jurkat E-6) or Monocytoid cell line (U937) or PBL activated for 3 days with PHA+IL-2 were incubated with or without IgM (1 to 30 μg/1×10⁶ cells/ml) in PBS buffer containing CaCL₂ at 37° C. for 45 min, and without a wash step, cells were re-incubated at 37° C. for 45 min with biotin labeled cytokine (50 ng). Cells were then re-washed in the cold and stained with PE-streptavidin.

Immunoprecipitation Technique and Western Blot Procedure to Detect IgM Binding to Solubilized Cell Membrane Receptors

Cell lines (80×10⁶) were incubated for 30 min at 4° C. with 10 ml of 100 mM (NH4)₂SO₄, 20 mM Tris HCl (pH 7.5) containing 10% glycerol, 1% Cymal-5 (Anatrace, Maumee, Ohio) and 1 tab mini-complete (Roche) to solubilize membrane receptors with minimal denaturation. IgM/receptor complexes were formed by interacting 100 μl of cell lysate (containing the equivalent of 50×10⁶ cells) with 100 μg of IgM. The mixture of IgM/cell lysate was then interacted with 50 μl of washed Agarose bead pellets containing covalently bound goat IgG anti-human IgM (Sigma). The agarose bead with bound IgM/receptor complexes was washed x3 (700 rpm) with buffer containing 1% bovine albumin and x2 with buffer alone. The washed beads with IgM/receptor complexes were then interacted with Laemmli buffer containing 4% 2-ME and incubated at 37° C. for 30 minutes to dissociate and linearise receptors under minimal reducing conditions. Incubating at higher temperatures led to dissociation and denaturation of the goat IgG (covalently bound to the Agarose bead). Supernatants were then electrophoresed in SDS-PAGE and transferred on to nitrocellulose and then probed with primary IgG antibodies specific for the receptor in question. It was not unusual for the secondary HRP conjugated antibody (even if specific for the primary mouse or rabbit Fc fragment of IgG) to bind to extra protein bands of both the heavy and light chains of goat IgG (that disassociated from the beads) as well as the light chains of IgM. Hence the secondary antibody was routinely pre-absorbed with goat IgG and human IgM prior to use. In some experiments, we resorted to using unlabeled secondary antibody specific for goat IgG (H & L), especially if the primary antibody was of non-goat origin. Additionally as negative controls, the Western blot procedure was performed with supernatant from beads that were interacted with IgM (but with no cell membrane lysate) or with beads that were interacted with lysate (but with no IgM) so as to identify presence of non-specific bands. As a positive control the membrane lysate without beads or IgM was interacted with Laemmli buffer under similar conditions and then electrophoresed in SDS-PAGE.

ELISA Assay to Detect IgM or IgG Binding to CD4 or CD81

We employed previously described techniques. 100 l containing 250 ng of recombinant, full length extracellular domain of CD4 (Progenics) or CD81 (a gift from Dr. Shoshana Levy) were added to a 96 well nunc-immulon plate and the ELISA plate was prepared using the Immuno-Tek ELISA construction system (Zepto Metrix, Buffalo, N.Y.). 200 to 300 ng of IgM from serum, culture supernatants or purified IgM was added to the well and incubated at RT for 1 hour. The secondary antibody used was a 1:4000 dil of HRP goat anti-human IgM or IgG (Southern Biotech, Birmingham, Ala.).

Antibodies for Western Blots

The following antibodies were used as primary antibodies in the Western blot procedure: Polyclonal IgG rabbit antibodies to IL2-R (α or β chain), CD3, CD4, HLA-A, HLA-DR, or CXCR4; monoclonal mouse IgG antibodies to CCR5 (clone CTC, N-terminal) and CXCR4 (clone 4G10 N-terminal). Antibodies were obtained either from R & D Systems, MN, or Santa Cruz Biotechnology, CA or Biochain Institute, CA. The following HRP conjugated secondary antibodies (Fc fragment specific) were used: polyclonal IgG goat antibodies to rabbit IgG, mouse IgG, or human IgM. All secondary antibodies were obtained from Jackson Immunological Labs.

Chemotaxis Assay

This assay was performed using the 24 well Costar transwell tissue culture inserts with 5 micron polycarbonate filters. 0.15×10⁶ cells in 0.15 ml RPMI with 0.5% human albumin were added to the upper transwell. Thirty minutes later 100 ng of SDF-1α or RANTES or MIP-1α were added to the bottom well containing 0.6 ml of the same media as in the upper well. The chemotaxis assay was performed at 37° C. for 2 hours for activated PBL, 4 hours for Jurkat cells and 12 hours for Hut78 cells. Cells migrating to the bottom well were enumerated by flow cytometry. Chemotaxis index was calculated by dividing the total number of cells migrating in presence of chemokine by the number of cells migrating in the absence of chemokine. As a control for chemotaxis, four-fold chemokine was added to the upper transwell in presence or absence of chemokine in the bottom well. The effect of IgM on chemotaxis was evaluated by incubating IgM (5 to 30 μg/ml) with cells at 37° C. for 30 min prior to adding cells to the upper transwell.

MLR Assay

Briefly, 0.15×10⁶ PBL in 0.15 ml RPM1 containing 10% fetal calf serum were co-cultured (in triplicate) in flat bottom wells with similar number of cells from another individual known to have different HLA-Class 1 and DR antigens. After 5 to 6 days in culture, [H]³ Thymidine was added to cells in each well of a 96 well plate and 12 to 18 hours later cells were harvested over a filter matrix and the uptake of Thymidine by proliferating cells was quantitated using a liquid scintillation counter. Different doses of IgM was added on Day 0 and Day 1 of the culture period.

Quantitation of Cytokines in Culture Supernatants

Cytokines in PBL culture supernatants were assayed in a semi-quantitative manner using the Ray Bio Human cytokine Array #3 kit (Ray Biotech, GA) which consists of a membrane array containing 42 different primary murine antibodies, each specific for a cytokine. One ml of supernatant is incubated for 2 hours with the membrane which is then washed and re-incubated for one hour with a cocktail of the same 42 primary antibodies. After re-washing, the membrane is incubated with an HRP conjugated secondary antibody. Cytokine positive spots are detected on an X-ray film and quantitated with a densitometer. Significant changes in cytokine levels as detected by the Ray Bio assay was confirmed and quantitated with an ELISA technique.

Quantitation of Intracytoplasmic Cytokines

Certain cytokines e.g. IL-2 and IFN-γ, could not be detected by the Ray Biotech kit either because these secreted cytokines avidly bind to cell receptors (e.g. IL-2) or are secreted at very low levels in the MLR culture. To circumvent this problem we evaluated by two color flow cytometry the percent of activated T cells expressing intracellular cytokine. Briefly, PBL were either activated in an MLR or by immobilized anti-CD3 and cells were assayed for intracellular cytokine on Day 4 of culture. Brefeldin (10μg/ml) was added 12 to 14 hours prior to terminating the culture. Murine monoclonal antibodies, specific for the above cytokines, and effective for intracytoplasmic staining after cell fixation, was obtained from eBioscience. Maximum percent of T cells with intracytoplasmic cytokines was observed at Day 3 or Day 4 of culture.

Quantitating Phosphorylation of Intra-Cellular Zap-70

Studies on phosphorylation of Zap-70 were performed with freshly obtained PBL and phosphorylation was quantitated at 0, 2, 5 and 10 mins (early stage) or at 16 hrs (late stage). In these studies, cells (0.6×10⁶/0.6 ml) were initially incubated with or without IgM (final conc 5 to 15 μg/ml) for 30 to 45 min at 37° and were then activated with immobilized anti-CD3 (OKT3) (1 μg of antibody in a well of a 48 well plate). Cells were then incubated for the required time at 37° in RPMI media with HEPES buffer and no fetal calf serum (FCS) for the “early stage” experiments and in the same media with 5% FCS and in 5% CO₂ for the “late stage” experiments. Phosphorylation of Zap-70 was evaluated in the absence (to determine background phosphorylation) or presence of immobilized anti-CD3. PBL activated for the desired length of time were immediately chilled in ice for 10 mins prior to fixing and permeabilisation. Cells were then stained with antibodies for the phosphorylated signaling protein or for the total signaling proteins and antibody binding to the signaling protein was quantitated by flow cytometry.

Temperature Dependence for the Cytolytic Effects of IgM Anti-leukocyte Antibody

Temperature dependence for the cytolytic effects of IgM anti-leukocyte antibody is evaluated by a complement dependent microlymphocytotoxicity assay. Various concentrations of IgM antibody are reacted for 1 hour with either 2×10⁵ PBL or IL-2-activated PBL (7 days) before adding fresh rabbit serum as a source of complement. After about 2 hours, the cells are washed twice before adding trypan blue and enumerating dead cells that stain blue. Experiments are performed at 15° C. and 37° C.

IgM Inhibition of HIV-1 Infection of Cells

a) HIV-1 Infection of GHOST Cells

It has been observed that the HW-1 R5 virus utilizes CCR5 receptors for cell entry, while the HIV-1 X4 virus uses CXCR4 receptors. Studies are conducted, therefore, to determine whether IgM inhibits HIV-1 entry into cells in light of such observations.

In these studies, GHOST CCR5 and GHOST CXCR4 transfectant cell lines are infected with HIV-1. The GHOST cells are derived from HOS cells transfected with either CCR5 or CXCR4 genes and also co-transfected with the HIV-2 LTR driving hGFP construct. The hGFP construct enables cells infected with HIV-1 virus to emit a green fluorescence so that the number of infected cells can be quantified using flow cytometry. These cell lines are particularly suited for these studies because single-cycle viral replication can be detected in less than 48 hours.

About 2×10⁴ each of GHOST CCR5 and CXCR4 cells are separately cultured for about 12 hours in about 1 ml RPM1 media containing about 10% fetal calf serum in a 12-well plate. Normal, HIV or ESRD IgM is then added to each of the GHOST CCR5 and CXCR4 cells about 30 minutes prior to adding the R5HIV-1 virus to GHOST CCR5 and the X4 HIV-1 virus to GHOST CXCR4. Both virus and antibody are present throughout the 48-hour culture period. No polybrene is used to enhance viral entry into the cells.

After the 48-hour incubation period, cells are harvested and fixed in formalin. Infected cells emitting green fluorescence are enumerated with flow cytometry.

Additionally, similar data is obtained when the virus or IgM antibody is washed about 4 hours after incubating with GHOST cells.

b) HIV Infection of Activated Human PBL

Human PBL are pre-treated with Phytohemmaglutium (PHA-P) and IL-2 to increase receptor expression (e.g. CCR5, CD4) on T lymphocytes and monocytes as well as to activate such cells, both of which enhance HIV-1 entry and replication. Therefore, Ficol/Hypaque separated PBL (2×10⁶ cells per ml in culture media containing 10% fecal calf serum) are pretreated with PHA-P (5 mg/ml) and IL-2 (40 units/ml) and cultured for 24 to 48 hours in 5% CO₂. Cells are washed prior to adding IL-2, IgM and the HIV-1 virus. The cells are not washed any more but are kept growing for 12 to 14 days. On day 7, half the culture supernatant is removed (and saved) and the culture well is supplemented with 1×10⁶ freshly activated PBL (48 hour old) and also replenished with half the quantity of IgM and IL-2. On day 12 to 14 culture supernatants are harvested and p-24 core antigen in culture is quantitated using and ELISA technique.

c) HIV-1 Infection of Human PBL/SCID Mice

We employed (with modifications) the procedure developed by Mosier and as described in Torbett et al, Immunol Reviews 124: 139-164, 1991, which is incorporated herein by reference. Seven to eight week old female CB17 SCID mice, purchased from Harlan Sprague Dawley, Indianapolis, Ind. and having <1 μg per ml of mouse IgM in their plasma were injected intraperitoneally with freshly isolated 25−35×10⁶PBL in 1 ml RPMI containing 10% FCS and antibiotics (RPMI culture media). Two hours later mice were re-injected intraperitoneally with 10⁵ TCID₅₀ HIV-1 virus in 1 ml RPMI culture media. One ml of IgM at 1 mg/ml, obtained from the same PBL donor, was injected intraperitoneally, either immediately after the HIV-1 injection or 48 hours later. The same dose of IgM was injected every five days until day of sacrifice as kinetic studies revealed that human IgM in mouse plasma attained peak levels of 40-50 μg by day two and 8-10 μg per ml by day five after the intraperitoneal dose. Mice were sacrificed three weeks after the human PBL injection. Percent human CD3 and CD4 positive T lymphocytes in spleen cells were quantitated with FITC labeled mouse anti-human CD3 or CD4 (BD Pharmigen) using flow cytometric techniques. Secondly, murine spleen cells were co-cultured with two day old day IL2-activated autologous PBL to quantitate HIV-1 in spleen cells. In co-culture studies 2×10⁶ spleen leucocytes in 1 ml RPMI culture media were co-cultured with 2×10⁶ PHA+IL-2 activated (2 days old) human PBL in 1 ml RPMI culture media containing human IL2 (30 units/ml). Co-cultures were fed at weekly intervals with two-day-old 2×10⁶ IL2-activated autologous PBL. p24 antigen in co-culture supernatants was quantitated after two and three weeks of co-culture using an ELISA kit. With this protocol (i.e. single dose of virus and sacrifice at three weeks) one could not detect viremia after the first week. Studies on SCID mice were approved by our Institutions Animal Care and Use Committee.

d) Inhibition of Syncytia Induced by X4-IIIB

The T cell line (Sup T-1), when infected with the X4-IIIB virus, spontaneously forms multi-nucleated giant cells termed “syncytia”. Syncytia are formed when the viral envelope, expressed on the cell-membrane, binds to CD4 and CXCR4 of an adjacent cell thus leading to fusion of cells. In this assay, infected Sup T-1 cells (forming 1 to 2 syncytia per HPF×400 magnification) were co-cultured with un-infected Sup T-1 cells at a ratio of 1 infected to 10 uninfected cells. Formation of new syncytia was evaluated at 24 to 48 hours. In the inhibition assays, IgM (10 g/ml) or soluble CD4 (20/ml) was initially added to uninfected cells at 37 C for 1 hour following which co-cultures were set up in 24 well plates with 1×10⁶ cells/ml.

e) IgM Mediated Inhibition of Pseudotyped HIV-1 Viral Envelopes

IgM mediated inhibition was tested using a single-round viral entry assay. Pseudotyped HIV-1 expressing the firefly luciferase gene and containing various HIV-1 envelope glycoproteins was produced using previously published techniques (See Yang X et al J of Virology, 75, 1165-1171, 2001). The envelope glycoproteins used to construct pseudovirus includes JR-FL and ADA, which use CCR5 as the co-receptor for entry; HXBc2, which uses CXCR4 as the co-receptor, and the glycoprotein of vesicular stomatitis virus (VSV-G), which is believed to use phospholipids as the receptor for entry. The target human PBL were pre-activated with PHA and IL-2 for 48 hours. 1.2×10⁵ PBL in triplicate were incubated with or without 5 mg IgM prior to the addition of pseudotyped HIV-1. The mixture was incubated at 37° C. in 5% CO₂ for 2 days, after which the medium was aspirated and the luciferase activity in the target cells was measured using the GloMax luminometer (Promega).

Method of Cardiac Transplant, Diagnosis of Rejection and Histological Score

Intra-abdominal Cardiac Transplant Procedure: This was performed on 8-12 week old donor and recipient male mice. Through an abdominal incision, the donor heart aorta was anastomosed to the recipient infra renal aorta and the donor heart pulmonary artery was anastomosed to the inferior vena cava.

Histological Score Cardiac allografts were analyzed in a blinded fashion. Parenchymal rejection was graded using a scale modified from the International Society of Heart and Lung Transplantation (0=no rejection; 1=focal mononuclear cell infiltration without necrosis; 2=focal mononuclear cell infiltration with necrosis; 3=multifocal mononuclear cell infiltration with necrosis; 4=widespread infiltrates with hemorrhage and/or vasculitis).

Results

Introduction

Data in the result section will be presented in the following order:
a) Studies to show that IgM binds to Lymphocytes, other leucocytes and malignant cells and studies to show that IgM does not cause complement mediated cell lysis of normal cells at 37° C.
b) Studies to show that purified serum IgM inhibits HIV-1 infection (i) in-vitro and (ii) in-vivo.
c) Studies to show that IgM inhibits T cell proliferation and chemotaxis.
d) Studies to determine some of the mechanisms for IgM inhibition of T cell activation, proliferation and cytokine production including (i) immunoprecipitation studies to show that IgM binds to CD3 and CD4 and (ii) studies showing that IgM down-modulates CD4 and CD2 receptors, (iii) studies showing that IgM inhibits proximal intracellular events activated by the T_{c}R/CD3 receptor and (iv) studies showing that IgM inhibits secretion of certain cytokines e.g. TNF-α and IL-13.
e) Studies to determine some of the mechanisms for IgM inhibition of chemotaxis including (i) immunoprecipitation studies to show that IgM binds to CCR5 and CXCR4, (ii) studies showing that IgM inhibits binding of MIP-1α and RANTES to CCR5 and inhibits binding of SDF-1α to CXCR4, (iii) studies showing that IgM down-modulates CCR5 but not CXCR4, and (iv) studies showing that IgM prevents chemokine induced internalization of CXCR4.
f) Summary of above data delineating mechanisms for IgM mediated inhibition of HIV-1
g) Studies to show that IgM anti-lymphocyte autoantibodies inhibit the inflammatory response mediated by an allograft (i.e. rejection) in kidney transplant recipients and in an IgM knockout mouse model.
h) Studies to show that IgM anti-lymphocyte autoantibodies cause cell death of lymphoma cells at 37° C.
i) Studies to show that IgM-ALA are naturally occurring and that naturally occurring monoclonal IgM-ALA will bind to CD4 and inhibit HIV-1.
j) Studies showing that IgG-ALA are absent in newborns and in normal sera.
k) Studies to show that IgM-ALA bind to CD81 and inhibit hepatitis C infectivity of cells.
l) Studies to show that the inhibitory effect of IgM-ALA is not affected by human serum.
m) Studies to show that LPS enhances IgM-ALA in normal mice without causing auto-immune disease.
   Presentation of Data
   a) IgM Binds to Lymphocytes, Other Leucocytes and Malignant Cells and does not Cause Cell Lysis at 37° C. of Normal Cells
   (i) Binding of IgM to Lymphocytes, other Leukocytes and Malignant Cells

In these studies flow cytometric techniques were used to quantitate binding of IgM to the different cells. As seen in FIGS. 1A and 1B, Normal IgM, and HIV IgM contain IgM antibodies that bind to Sup T-1 (FIG. 1A) and GHOST CD4-CXCR4 cells (FIG. 1B). As seen in FIGS. 1D and 1E, Normal and HIV IgM contains antibodies that bind to T-lymphocytes isolated from peripheral blood (FIG. 1D) and neutrophils isolated from peripheral blood (FIG. 1E). The negative control in each figure indicates that no IgM was incubated with the various cells. Similar data were obtained with the addition of normal, ESRD, HIV-1 and Hepatitis C sera to the different cells. All these sera had demonstratable binding of IgM to autologous and allogeneic cells including non-lymphocyte leucocytes, and malignant cell lines.

(ii) Non-lytic Nature of IgM Anti-lymphocyte Antibodies at 37° C. Using Normal Cells

About 40 to 60% cell lysis of normal lymphocytes was observed in presence of complement when the assay was performed at 15° C. Higher levels of cell lysis was observed with IL-2-activated lymphocytes, which have increased expression of receptors. IgM, when used at amounts of about 1.0 microgram or more, caused cell lysis, while CK15 lysed cells at concentrations of about 0.05 microgram or more. When the assay was performed at 37° C., however, less than about 5% lysis was observed with normal or IL-2 activated lymphocytes. These observations are in agreement with several reports clearly demonstrating that IgM anti-lymphocyte autoantibodies are lytic at colder temperatures but not at 37° C. (See Lobo P I et al in Lancet Vol 2, p 879-83, 1980).

b) Studies to Show that Purified serum IgM Inhibits HIV-1 Infection of Human PBL

(i) In-Vitro Studies to Show that Human IgM Inhibits HIV-1 Infection of PHA+IL-2 Activated Human PBL.

IgM, in these studies, were obtained from sera of normal individuals, HIV-1 infected individuals and ESRD patients awaiting kidney transplantation. Some of the HIV-1 infected patients had AIDS while others were long term non-progressors. In these studies, IgM from longterm non-progressors will be referred to as HIV-IgM while IgM from AIDS patients will be referred to as AIDS IgM. We used ESRD IgM to compare if IgM-ALA that develops as part of an inflammatory response in ESRD or after HIV-1 infection have similar inhibitory activities as predicted by our hypothesis. In these studies we also used IgM purified from serum of rats, mice, goats, and rabbits. In the initial studies different HIV-1 strains (X4 and R5) were used to infect 48 hour mitogen activated PBL and using different concentrations of IgM, all in the physiological range. Maximal inhibitory activity was noted with IgM preparations at 15 or more μg/ml although with certain viral strains near maximal inhibition was seen with IgM as low as 4 μg/ml. IgM from 4 to 5 individuals were pooled due to insufficient quantity but in certain experiments, (where indicated) IgM, from single individuals were used and there was no difference in the overall results. Data from 5 different experiments are presented in Table I and Table II using an R5 strain (8658) and the X4 strain IIIB. Interestingly, Normal, HIV, and ESRD IgM, as well as sol CD4 inhibited viral replication of the 8658 (R5) and the IIIB (X4) strains by more than 98%. All AIDS IgM had similar levels of IgM-ALA as HIV-IgM when evaluated by flowcytometry but 5 of 7 AIDS IgM failed to inhibit HIV-1 in these in-vitro assays indicating that there is heterogeneity in IgM-ALA antibodies and that AIDS IgM lacks certain specific IgM that binds to receptors important for viral entry. All animal IgM had the most inhibitory effect on all three HIV-1 viral stains. Monomeric normal human IgM had similar inhibitory effect on HIV-1 infectivity as the pentameric form of IgM. Pooled human IgG that was isolated from 20 different normal sera had no inhibitory activity on 4 different HIV-1 isolates when used at 50 μg/ml. However >50% inhibition of some HIV-1 isolates was observed when pooled IgG, isolated from HIV sera, was used at concentrations of 100 to 200 μg/ml.

TABLE I
Effect of purified IgM from normal, HIV, and ESRD
individuals on in-vitro infectivity of HIV-1 virus

Pg/ml of p24 core antigen

| | IIIB(X4) | HIV-1 8658(R5) |
|---|---|---|
| Media | >32,813 | 34,602 |
| Normal IgM | 1,514 | 579 |
| HIV IgM | 439 | 672 |
| ESRD IgM | 870 | 230 |
| Waldenstrom IgM | >32,813 | 32,700 |
| Autologous Human serum | ND | 4,249 |
| RANTES (500 ng) | ND | 11,246 |
| Sol-CD4-183 (20 μg) | ND | 3,949 |
| pool Human IgG (50 μg) | >32,813 | ND |

Table I - Data are representative of 5 different experiments. Each p24 value is a mean of triplicate cultures with less than 15 percent variation from the mean. In these studies IgM used were from a pool of 3 to 4 different individuals and added 30 minutes before the virus. ND = Not Done

TABLE II
Summary of all in-vitro studies evaluating percent inhibitory
effect of Normal, ESRD, and HIV-1 IgM on different HIV strains
Mean of Percent Inhibition

| | IIIB (X4) | 8658 (R5) |
|---|---|---|
| Normal IgM | (3) 97.6 ± 1.7 SD | (5) 98.6 ± 0.9 SD |
| ESRD IgM | (3) 99.2 ± 1.0 SD | (4) 99.1 ± 0.6 SD |
| HIV IgM | (3) 99.4 ± 0.5 SD | (4) 99.4 ± 0.9 SD |

(N) indicates number of different experiments, each done in triplicate. P-24 levels in viral cultures without IgM varied from 29,000 to 200,000 pg/ml

We next studied the kinetics of the inhibitory effect of IgM. As depicted in FIG. 2, purified normal IgM inhibited HIV-1, IIIB(X4) as well as 8658 (data not shown) even when added 96 hours after initiation of the viral cultures. These findings prompted us to determine if there was anti-viral activity in non-IgM-ALA antibodies.

To exclude this possibility, IgM was initially absorbed with the Jurkat T cell line and the U937 monocytoid cell line to remove IgM with binding to CD3, CD4, CXCR4, and CCR5. As seen in Table III, the inhibitory activity of IgM on HIV-1 infectivity was removed after absorbing with the U937 and T cell line, thus indicating that the inhibitory activity on HIV-1 resides in IgM that binds to leucocytes (i.e. IgM-ALA). The data thus far suggested that IgM-ALA could inhibit HIV-1 by inhibiting viral entry. Two approaches were used to determine if IgM inhibits HIV-1 viral entry. Firstly we resorted to the GHOST CCR5 and GHOST CXCR4 tranfectant cell lines to verify that IgM inhibits viral entry. These cell lines are stably co-transfected with the HIV-2 LTR driving hGFP construct, which emits a green fluorescent upon integration of HIV-1 viral genome into the cell DNA. Hence one can measure entry efficiency of the virus especially if cells are harvested in 48 hours, which allows for a single cycle of viral replication. Data from FIG. 3A clearly demonstrates that in the presence of IgM, viral entry is reduced by more than 95%. In a second set of experiments we wanted to determine if IgM inhibits viral entry by inhibiting attachment of HIV-1 to coreceptors. Multinucleated giant cells termed “syncytia” are formed when the HIV-1 viral envelope, expressed on the cell-membrane of an infected cell, binds to co-receptors of an adjacent cell thus leading to fusion of cells. Data in FIG. 3B demonstrates that soluble CD4 and HIV-IgM (but not Waldenstrom IgM) clearly inhibited syncytia formation as a result of inhibiting attachment of HIV-1 gp120 on to co-receptors. Conversely, absorption of IgM anti-CD4 from HIV-1 IgM removed the inhibitory effect on syncytia formation. Data in FIG. 3C demonstrates that normal IgM inhibits binding and entry of pseudotyped HIV-1 viral envelopes from different HIV-1 strains into activated PBL (please see methods for details).

TABLE III
Experimental details as in Table I. IgM was added on Day 0. Details
of IgM absorption with Jurkat and U937 cells are in section of
“Methods of Procedure”. Representative data from two separate
experiments are presented. Data are mean of triplicates with
less than 10 percent variation from the mean

p24 antigen (pg/ml)

| | IIIB(X4) | 8658(R5) |
|---|---|---|
| Media | 14,849 | 23,525 |
| Normal pool IgM | 2,134 | 581²⁵ |
| (16 μg/ml) | | |
| Normal pool IgM | 13,107 | 28,061 |
| (16 μg/ml) absorbed | | |
| with Jurkat and | | |
| U937 cells | | 30 |

TABLE IV
Experiments to determine if normal pool IgM inhibits X4 and
R5 HIV-1 viral strains in an in-vivo PBL-SCID mice model

| | # of mice infected at 3 weeks | |
|---|---|---|
| | 8658(R5) HIV-1 virus | IIIB(X4) HIV-1 virus |
| PBL | 0/4 | 0/4 |
| PBL + HIV | 10/15 (66%) | 3/4 (75%) |
| PBL + HIV + IgM | 3/11 (27%)* | 1/7 (14%) |

(ii) Studies Show that Normal Igm Inhibits In-Vivo HIV-1 Infection in a Human PBL-SCID Mice Model

We used this well described in-vivo model to confirm observations with the in-vitro PHA+IL-2 activated PBL assay. The PBL in this model are not pre-activated with mitogen prior to viral infection and hence the inhibitory effect of IgM-ALA on T cell activation can also play a role in controlling viral replication. Details of the experimental method and quantitation of IgM levels in the serum are described in section on “methods of procedure”. Studies were not done with HIV and ESRD IgM as it was difficult to obtain blood in quantities needed for these experiments. Data with pooled normal IgM and the two different HIV strains are depicted in Table IV. These preliminary data bring out two observations. Firstly, 30 percent of infected mice can spontaneously become non-infected because of CD4 cell depletion, and this observation was also noted by Mosier. Hence at 3 weeks 60-70% of mice remained infected. However, normal IgM reduced the number of infected mice to 27% and this difference with the 8658(R5) virus was approaching statistical significance (p<0.08). There were too few mice used with the IIIB but again IgM reduced the number of mice that were infected from 75% to 14%. However, if one combined data of both 8658 and IIIB, then clearly the decrease in infected mice with IgM was statistically significant (p<0.05) (Fishers Exact Test). The decrease in HIV-1 infection of human-PBL-SCID mice in the presence of human IgM was not due to IgM or HIV-1 depletion of human PBL as by three color flow cytometry we could not detect significant changes in the splenic human T cell population (CD45+, CD4+, CD4+) between SCID mice treated with IgM+HIV+PBL and control SCID mice treated with PBL.

In summary, these data clearly showed that IgM obtained from normal, ESRD, and HIV-1 infected patients inhibits HIV-1 from infecting activated human PBL in-vitro and in-vivo and this inhibitory effect is removed after absorbing IgM with the U937 monocytoid line and the Jurkat T cell line indicating that inhibition of HIV-1 infectivity is mediated by IgM that binds to cell coreceptors important for HIV-1 viral entry into cells. Additionally, experiments depicted in FIG. 3 indicate that the inhibitory effect of IgM is mediated by blocking the binding of the HIV-1 virus or their pseudotyped envelopes on to cell membrane receptors. Even though, AIDS IgM has high levels of IgM-ALA, the majority of purified AIDS IgM (unlike HIV-1 IgM) failed to inhibit several HIV-1 isolates, indicating therefore that AIDS IgM, lacks IgM that binds to receptor epitopes important for HIV-1 viral entry. Our findings in addition, cannot be explained on IgM with reactivity to Tat and gp120, which may be present in the purified IgM preparations as previous investigators have shown that IgM with anti-Tat and anti-gp120 do not have HIV-1 neutralizing activity and do not inhibit viral entry into cells. Similarly, our findings cannot be explained on IgM neutralizing the HIV-1 virus as there is prior art to show that fresh human serum and IgM (including naturally occurring IgM) with activity to HIV gp120 does not lyse or inactivate the HIV-1 virus. Additionally, prior art shows that HIV-1Tat protein induces apoptosis of T cells and that naturally occurring IgM anti-Tat inhibits HIV-Tat induced T cell apoptosis. (See Rodman T C et al, Exptl Hematology, Vol. 29, p. 1004-1009, 2001; Berberian et al Science Vol 261 p 1588-1591, 1993; Llorente M. Scand J of Immunol, Vol 50 p 270-279, 1999; Hoshino H, Nature Vol 310 p 324-325, 1984; and Bonapur B, Virology Vol 152 p 268-271, 1986 for prior art in this regard. We could not detect RANTES or SDF-1α in these IgM preparations using ELISA and Western blot techniques.

The increase in IgM-ALA to diverse infective and inflammatory processes and the inhibition by IgM-ALA of HIV-1 infectivity prompted us to evaluate whether IgM-ALA mediates this inhibitory effect by binding to receptors needed by the HIV-1 virus for cell entry as well as receptors involved in inflammation. Binding of IgM to T cell receptors and to chemokine receptors appeared to be an attractive possibility. We initially examined these possibilities by determining if IgM purified from serum (i) inhibited alloantigen (MLR) and anti-CD3 induced T cell proliferation and (ii) inhibited chemotaxis in response to chemokines. In these studies, we compared normal IgM with HIV-1 and ESRD IgM. Waldenstrom IgM was used as a negative control in these studies.

c) Studies to Show that IgM Inhibits T Cell Proliferation and Chemotaxis

(i) IgM Inhibits MLR-Induced Proliferation

An MLR assay (see methods) was used as an initial step to evaluate the effect of IgM on T cell proliferation in response to alloantigens. As can be seen from FIG. 4, pooled ESRD IgM, but not pooled normal and HIV IgM, significantly inhibited T cell proliferation using physiological doses of IgM i.e. 15 μg/ml. ESRD IgM failed to inhibit T cell proliferation when added after 24 hours of culture. Pooled normal IgG (50-100 μg/ml) or albumin had no inhibitory effect in the MLR assay. Normal non-pooled IgM inhibited MLR when used at 30 to 40 μg/ml. Pooled normal IgG inhibited MLR when used at doses of 120-200 μg/ml. To determine if the observed effect of ESRD IgM was due to IgM that bound to T cells, we absorbed ESRD IgM with the U937 and Jurkat T cell line (see methods) to remove any IgM anti-leucocyte reactivity. IgM absorbed with these cell lines failed to inhibit T cell proliferation in the MLR assay clearly indicating that the observed inhibition of T cell proliferation with ESRD IgM was due to IgM that bound to leucoyctes.

(ii) IgM Inhibits Anti-CD3 Induced T Cell Proliferation

We wanted to determine if IgM affects anti-CD3 induced proliferation of PBL. In these studies normal PBL (3×10⁵ in 0.3 ml) were exposed to 0.01 μg OKT3 (a murine IgG2a anti-CD3 monoclonal) and then cultured for 4 days in 96 well flat bottom plates prior to determining extent of cell proliferation using H³-labeled thymidine. Pooled normal, HIV IgM, or ESRD IgM (15 μg) was added to these cell cultures at initiation of the culture. Data from one of 3 experiments is depicted in FIG. 5. HIV and ESRD IgM significantly suppressed anti-CD3 mediated proliferation of T cells. Again ESRD IgM failed to inhibit T cell proliferation when added after 24 hours of culture. Normal non-pooled IgM inhibited anti-CD3 induced T cell proliferation. These data are similar to those observed with the MLR induced T cell proliferation (See FIG. 4).

(iii) IgM Inhibits Chemotaxis

We wanted to determine if IgM inhibits chemotaxis of activated PBL and T cell lines in response to SDF-1α. All IgM preparations inhibited chemotaxis. However ESRD IgM had a significantly more pronounced inhibitory effect on chemotaxis as depicted in FIG. 6 for PBL.

These differences in inhibitory effects on chemotaxis were not due to increased apoptosis or cell death as evaluated by flow cytometry using propridium and anti-annexin and would suggest that ESRD IgM in addition inhibits chemotaxis through effects on other cell receptors (e.g. adhesion molecules or integrins) and/or intracellular activation pathways that are involved in both chemokinesis and chemotaxis activity. However, ESRD IgM has a more pronounced effect on chemotaxis when compared to normal IgM, suggesting that ESRD IgM may in addition inhibit intracellular activation pathways involved in chemotaxis. Pooled normal IgG at doses of 50-100 μg/ml did not inhibit chemotaxis.

d) Studies to Determine Mechanism for IgM-ALA Inhibition of T Cell Proliferation

Inhibition, especially by ESRD IgM, of T lymphocyte proliferation in response to alloantigens or anti-CD3 prompted us to determine if the inhibitory effect mediated by IgM was secondary to binding of IgM to TcR/CD3 and/or the co-stimulatory molecules. In support of such a concept are studies showing that binding of antibodies to the CD4 receptor, inactivates T cell proliferation in response to alloantigens or anti-CD3. Additionally there are studies to show that binding of antibody to CD3 (e.g IgG anti-CD3) inhibits T cell proliferation in response to alloantigens (MLR). We also wanted to determine if binding of IgM to the receptor resulted in down-regulation of the receptor. In these studies we used IgM purified from individual normal sera and compared to IgM obtained from individual HIV and ESRD IgM. These purified IgM preparations were used to immunoprecipitate different receptors from whole cell lysates of cell lines constitutively expressing high levels of these receptors.

(i) Immunoprecipitation Studies and Inhibition of Murine IgG Monoclonal Antibodies to Show that IgM Binds to CD3 and CD4

Here receptors in whole cell lysates were immunoprecipitated with purified individual normal, HIV or ESRD IgM, and then subjected to SDS-PAGE gel electrophoresis under reducing conditions at 37° C. for 30 minutes with 2ME (see methods for details). Receptors immunoprecipitated by IgM were transferred on to nitrocellulose membranes prior to using murine monoclonal or rabbit IgG polyclonal antibodies as primary antibodies to identify these receptors. We used several controls to exclude the possibility of non-specific receptor binding to the bead (i.e. in absence of IgM). Representative data from 3 separate experiments involving identical quantities of normal, HIV IgM, and ESRD IgM as well as identical quantities of whole cell lysates are depicted in FIG. 7. The data clearly demonstrates that both normal, HIV, and ESRD IgM immunoprecipitated CD3 and the CD4 receptor. As a group, HIV-IgM appeared to immunoprecipitate more CD4, when compared to Normal or ESRD IgM. Waldenstrom IgM (labeled W) did not immunoprecipitate CD4. We next wanted to determine if human IgM antilymphocyte NAA inhibited the binding of murine IgG anti CD4 monoclonal antibodies. In these studies, PBL were initially interacted for 30 min with purified IgM or human serum at 4 C, then washed and re-interacted with fluorochrome labeled murine IgG monoclonals specific for CD4. We used a panel of different murine monoclonals reactive to CD4 (Leu3a, SIM2, Q4120 and MT310). Using flowcytometry, purified IgM from normals, ESRD and HIV-1 individuals inhibited by >50%, the binding of some murine IgG anti-CD4 e.g. Leu3a, SIM2, Q4120 but not MT310. Some sera from ESRD and HIV-1 patients also inhibited the binding of the same three murine IgG anti-CD4 antibodies. These studies indicate that IgM anti-lymphocyte NAA bind to certain specific epitopes on the CD4. We next wanted to determine if inhibition of proliferation by IgM was merely due to IgM binding to CD3 and CD4 (thus causing a perturbation in the formation of the immunological synapse) or did IgM in addition down-modulate the receptors especially in light of previous studies showing that cross-linking of CD3 can down-regulate CD4.

(ii) Studies to Show that IgM Down-Regulates CD4 and CD2 but not CD8, HLA, and Other Co-Stimulatory Molecules

In these studies we used the MLR assay to activate T cells. Different doses of normal IgM were added either at the initiation of MLR, on day 3 of culture or 2 hours prior to harvesting the cells on day 4 of culture. Day 4 MLR activated cells were analyzed using two color flow cytometry for T cell co-stimulatory molecules. We used either PE or FITC-labeled murine monoclonals specific for the different receptors. Representative data from 4 different experiments involving different combinations of individuals are depicted in FIG. 8. We noted that normal, HIV, and ESRD IgM, when added to MLR cultures, markedly inhibited the density of certain co-stimulatory molecules on the cell membrane e.g. CD4 and CD2 but had no effect on CD3, CD 28 and CD8 (FIG. 8). HIV, ESRD, and Normal IgM did not, however, down-regulate CD154, CD28, CD3, PDL-1, IL2-R, HLA-A, B, HLA-DR membrane receptors, as well as surface and intracytoplasmic CD152 receptors (data not shown). Other studies were performed to determine if IgM inhibits expression of co-stimulating molecules i.e. CD80 (B7.1) and CD86 (B7.2) present on antigen presenting cells. In these studies, we evaluated CD80 and CD86 expression on CD14 positive monocytes and macrophages present in the MLR assays except receptor density was evaluated at 24 hours of initiating the MLR culture. IgM markedly inhibited expression of CD86 (but minimally inhibited expression of CD80) on CD14 positive monocytes and macrophages as exemplified in FIG. 9 which depicts IgM inhibiting ESRD IgM on expression of CD86.

This inhibitory effect was not accompanied by increased apoptosis or cell death as measured by flow cytometry quantification of annexin expression and propidium iodide uptake by cells. The degree of inhibition for CD4 and CD2 was similar whether IgM was added on Day 0 of MLR or 2 hours before termination of the MLR culture. Secondly, there was no significant difference in level of inhibition between normal or HIV or ESRD IgM when used at doses varying from 10 to 30 μg/ml. No inhibition was observed at doses less than 5 μg/ml.

Further experiments were performed to investigate the mechanism for the inhibitory effect on CD4 and CD2. Firstly we wanted to determine whether the inhibitory effect in the presence of normal or HIV IgM was an “active” process or due to a “blocking” effect i.e. by IgM inhibiting the binding of the murine anti-receptor monoclonal antibody that is used to detect the receptor. IgM was added 2 hours prior to termination of MLR on Day 4 except an aliquot of cells was also incubated at 4° C. with IgM during the 2 hour period. In 3 separate experiments, there was no decrease in MCF of CD4 and CD2 when IgM was incubated with cells at 4° C. indicating therefore that the decrease in density of surface co-stimulatory receptors was due to an “active” process. Either there was internalization of receptors or active down-modulation of receptors at 37° C. in the presence of IgM. This question was analyzed using flow cytometry. In these studies, we focused mainly on CD4 expression as these receptors were highly expressed. Cells were initially exposed to PE-anti CD4 to stain for surface receptor and after washing the cells were permeabilized using the BD Pharmigen Kit and then re-exposed to PE-anti CD4 to stain for intracytoplasmic receptors. Data are presented in FIG. 10. Data indicates that IgM at 37° C. down-modulates both surface and intra-cytoplasmic CD4 receptors.

We next wanted to determine if down-modulation of both membrane and intracytoplasmic CD4 was secondary to cross-linking of CD3 by the pentameric IgM or possibly a direct effect secondary to binding of IgM to CD4. Two approaches were used. Firstly we used a human monocytoid cell line (U937) which expresses CD4 but has no CD3 receptor. Incubating U937 cells for 2 hours at 37° C. in presence of normal or HIV IgM led to a 50 to 55% reduction in expression of CD4 indicating that down-modulation of CD4 by IgM was independent of CD3. Secondly, MLR activated lymphocytes were incubated at 37° C. for 2 hours with either pentameric or monomeric IgM. Again use of monomeric HIV IgM led to down-modulation of CD4 indicating that cross-linking of the CD4 receptor was not essential for down-modulation.

(iii) IgM-ALA Inhibits Proximal Signaling Events Following T Cell Activation

Prior studies have shown that T cell activation mediated by TcR pertubation results in recruitment, phosphorylation and activation of Zap 70 (see Pullar C E, Scand J of Immunol, Vol 57, p 333-341, 2003 for prior art in this regard). We therefore, wanted to determine if IgM inhibits phosphorylation of Zap 70 induced by anti-CD3.

In these studies freshly obtained human peripheral blood lymphocytes (1×10⁶ cells/ml) were pretreated with immobilized anti-CD3 for 12 hours at 37° C. in 5% CO₂ and then examined for intra cytoplasmic phosphorylation of Zap 70 using flow cytometry. Intracytoplasmic phospho Zap 70 was quantitated by fixing and permeabilising the cells prior to interacting the cells with a polyclonal rabbit antibody to phospho Zap 70 (Upstate, Charlottesville, Va.). Purified IgM (30 μg/ml) from normal, HIV and ESRD patients was added to the cells half an hour prior to adding the cells to immobilized anti-CD3.

As can be seen in FIG. 11, there was increased phosphorylation of Zap 70 in human T cells activated with anti-CD3. However, pretreatment of T cells with normal or HIV IgM inhibited Zap 70 phosphorylation.

(iv) IgM Inhibits Secretion of TNF-α, IL-2 and IL-13

Further studies were performed to determine if the anti-proliferative effects of IgM-ALA were associated with a decrease in cytokine production. Supernatants from MLR cultures (Day 5 to 6) were assayed for different cytokines in a semi-quantitative manner using the Array III kit, which can detect cytokines in culture media at levels of 5 to 10 pg/ml (see methods for details). The Array III kit detected a significant increase in the secretion of IL-6, IL-8, IL-13, TNF-α, GMCSF, MCP-1, MIG, MDC, TARC, and GRO in the MLR supernatants. However, presence of IgM at the initiation of the MLR culture had no inhibitory effect on production of IL-6, IL-8, GMCSF, MCP-1, MIG, and GRO (see FIG. 12A). Conversely all IgM preparations, including normal IgM, significantly inhibited secretion of TNF-α, IL-13, MDC, and TARC (see FIG. 12A). Inhibition of TNF-α is particularly important as prior art has shown that inhibitors of TNF-α (e.g. antibodies to TNF-α) can suppress inflammation in patients with rheumatoid arthritis and Crohn's disease (see McDevitt H, Arthritis Research 2002, vol. 4 (Suppl), p. 5141-5152). The changes in cytokine levels were similar whether supernatants were assayed on Day 1, 2, or 3 of the MLR culture. Cytokine levels were maximal on Day 5 of MLR as exemplified for TNF-α in FIG. 12A. No IL-2, INF-γ, TGF-β, and IL-10 could be detected in the MLR supernatants using the Array III assay technique probably because these cytokines are taken up by the proliferating cells. To circumvent this problem, we resorted to determining if IgM inhibits intracytoplasmic cytokines. As depicted in FIG. 12B, IgM inhibited production of intracytoplasmic IL-2 but not INF-. These data provide more evidence indicating that IgM-ALA can inhibit T cell function in addition to proliferation.

In summary, normal, HIV, and ESRD IgM immunoprecipitate CD3 and CD4 receptors. IgM-ALA also mediates CD4 and CD2 receptor down-modulation, independent of CD3 and in additional IgM inhibits phosphorylation and activation of Zap 70. IgM in addition, inhibits secretion of certain cytokines—in particular IL-2, TNF-α and IL-13. All these mechanisms most likely contribute to IgM-mediated (i) inhibition of T cell activation and proliferation induced by alloantigenic stimuli (MLR) or anti-CD3 antibodies, and (ii) inhibition of HIV-1 infectivity of cells.

e) Studies to Determine Mechanisms for IgM-ALA Mediated Inhibition of Chemotaxis

In these studies we wanted to determine if inhibition of chemotaxis was secondary to IgM-ALA down-modulation of these receptors (from inhibition of T cell activation) or due to a direct “blocking” effect of IgM-ALA on the binding of chemokine to the receptor.

(i) Immunoprecipitation Studies and Inhibition of Murine IgG Monoclonal Antibodies to Show that IgM Binds to CCR5 and CXCR4

Initially, we wanted to determine whether IgM bound to the chemokine receptor. We approached this question by determining whether IgM could immunoprecipitate CCR5 and/or CXCR4 from whole cell lysates of the Daudi B cell line, which constitutively expresses high levels of CCR5 and CXCR4. Representative data from three separate experiments, using whole cell lysates and identical quantities of IgM obtained from three different normal individuals, pooled normal IgM (6 individuals), pooled ESRD IgM from 5 individuals, and five individual HIV IgM is depicted in FIG. 13. As depicted in FIG. 13, all three normal IgM individuals immunoprecipitated low levels of CCR5 while only one of five HIV individuals immunoprecipitated CCR5 suggesting that HIV-IgM, unlike normal IgM, has decreased IgM with binding reactivity to CCR5. ESRD IgM, on the other hand, immunoprecipitated several fold more IgM anti-CCR5 when compared to Normal IgM. Immunoprecipitation studies with CXCR4 were totally unexpected. Here four of the five HIV IgM and all of the ESRD IgM had IgM with a high level of binding reactivity to CXCR4. In summary, different individuals, whether normal or with disease, produce different levels of IgM with reactivity to CCR5 or CXCR4. Interestingly, disease processes can also alter IgM anti-CCR5 or anti-CXCR4 profile. HIV-1 infected individuals, in general, have low levels or lack IgM anti-CCR5, while ESRD individuals produce high levels of IgM with reactivity to both CCR5 and CXCR4. Waldenstrom IgM (labeled W) failed to immunoprecipitate CCR5 or CXCR4. The lane containing only lysate (Ly) in FIG. 13 clearly demonstrates that Daudi lysates contain the non-glycosylated 36-39 kDa isoform of CXCR4, which is expressed at high levels on the cell membrane and detected by the 4G10 and 12G5 murine monoclonals. No glycosylated 47 kDa isoform of CXCR4 was present in the Daudi lysate. Note, however, that Daudi lysate contained the glycosylated isoform of CCR5 (42-43 kDa) which was immunoprecipitated by IgM.

We next wanted to determine if IgM anti-lymphocyte NAA inhibited the binding of murine IgG anti-CCR5 or anti-CXCR4 monoclonal antibodies. We performed similar studies as described for inhibition of murine IgG anti-CD4. Again purified IgM from normals. ESRD and HIV-1 and certain ESRD or HIV-1 sera inhibited the binding (by >50%) of certain murine IgG anti-CCR5 (e.g. 2D7, CTC-5) or anti-CXCR4 monoclonals (e.g. 44716) but not other monoclonals (e.g. 12G5 or 44708). These studies indicate that IgM anti-lymphocyte NAA bind to certain specific epitopes on the CCR5 or CXCR4 receptors.

(ii) IgM Inhibits Binding of MIP-1α and SDF-1a to their Receptors

Since IgM immunoprecipitated CXCR4 and CCR5 from cell membranes, it became important to determine if IgM inhibited binding of chemokine to these receptors. Data in FIG. 14 clearly demonstrates that Normal IgM inhibited binding of biotin labeled MIP-Iα to CCR5 and SDF-Iα to CXCR4 present on two cell lines. IgM inhibited chemokine binding in a dose dependent manner as exemplified for binding of MIP-1α to U937 cells, SDF-1α to SupT-1 cells. Incubating cells with IgM and/or chemokine at 37° C. or 4° C. did not change the magnitude of the inhibitory effect of IgM on chemokine binding thus indicating that the IgM mediated inhibitory effect was not due to internalization of the receptor at 37° C. Waldenstrom IgM and pooled human IgG had no inhibitory effect on chemokine binding.

(iii) Studies to Show that IgM Prevents SDF-1α Induced Internalization of CXCR4

IgM down-modulated CCR5, but not CXCR4, and decreased chemotaxis in response to RANTES and MIP-1α, and SDF-1α. These observations prompted us to investigate whether IgM, after binding to CXCR4 prevents SDF-α induced internalization of CXCR4. Such an experiment was-possible, as IgM did not inhibit binding of murine IgM monoclonals that can be used to detect CXCR4 (e.g. 12G5). To study this question, Jurkat T-cells expressing CXCR4 were pretreated with ESRD IgM (pre absorbed with mouse IgG) at 37° C. for 30 minutes, not washed, and then cells were interacted with SDF-1α (100 μg) at 37° C. for another 30 minutes. Cells were then washed and interacted with FITC labeled 12G5. Data in FIG. 15 (panel B) clearly indicates the SDF-1α markedly reduces CXCR4 expression at 37° C. (secondary to internalization). However, pretreatment of cells with IgM (15 μg/10⁶ cells) inhibits SDF-α induced internalization of CXCR4 (panel C) Similar data were obtained with a SupT-1 T cell line and the RAJI B cell line.

In summary, IgM-ALA (i) strongly inhibits RANTES and MIP-1α binding to CCR5 and also inhibits SDF-1α binding to CXCR4, and (ii) binds to both CCR5 and CXCR4 receptors except there are major differences in the level of IgM anti-CCR5 and anti-CXCR4 among different individuals and between disease states i.e. HIV-IgM from most patients have decreased IgM anti-CCR5 but not anti-CXCR4 while ESRD IgM has high levels of IgM reactive to both CCR5 and CXCR4. These observations provide a mechanism for IgM mediated inhibition of HIV-1 infectivity.

f) Delineating Some Mechanisms for IgM Mediated Inhibition of HIV-1 Infectivity

These data highlight certain observations:

(i) IgM-ALA bind to CD3, CD4, CCR5, and CXCR4. However, there are major differences in the repertoire of IgM-ALA among individuals and between normal and disease states. For example, IgM from most normal individuals has low level of antibodies that bind to CCR5 and CXCR4 while many (but not all) HIV-1 infected individuals, have high levels of IgM with reactivity to CXCR4 and low levels of IgM with reactivity to CCR5. Conversely, ESRD IgM has high levels of antibodies to both CXCR4 and CCR5. Similarly, AIDS IgM, unlike HIV and Normal IgM lacks IgM-ALA that is inhibitory to HIV even though all these IgM preparations contains significant amount of IgM-ALA binding to leucocytes.

(ii) IgM-ALA (a) inhibits T cell proliferation in response to alloantigens and anti-CD3 antibodies, with ESRD IgM having the most inhibitory activity, (b) significantly down-modulates CD4, CD2 and CCR5 receptors (but not CD8, CD3 and CXCR4) and again ESRD IgM has the most down-modulating effect on these receptors.

(iii) IgM-ALA inhibits T cell activation as evidenced by decreased phosphorylation of Zap-70 and in addition IgM-ALA inhibits secretion of certain cytokines, in particular IL-2, TNF-α and IL-13, MDC and TARC.

(iv) IgM-ALA in physiological doses, inhibits HIV-1 infectivity of PBL both in-vitro and in-vivo. This inhibitory effect of IgM on HIV-1 appears to be mediated by an inhibitory effect on viral entry (see—FIG. 3) as well as on T cell activation. ESRD IgM which has high levels of IgM binding to CD4, CCR5, and CXCR4 has the most inhibitory effect. AIDS IgM has the least inhibitory effect on HIV-1 infectivity.

g) IgM Anti-Lymphocyte Auto Antibodies Inhibit Rejections in Kidney Transplant Recipients and in an IgM Deficient Mouse Model.

Since normal IgM inhibited the binding of chemokines (SDF-1α and RANTES) to their respective receptors and since ESRD IgM inhibited lymphocyte activation in a mixed lymphocyte culture (MLC), it became necessary to test whether in-vivo, there would be a strong correlation between the presence of high levels of these antibodies in the recipient and protection against kidney transplant rejections.

Accordingly, the level of IgM anti-lymphocyte antibody activity in the recipient was quantitated using flow cytometry to detect binding of IgM to donor T lymphocytes (see FIG. 16). Presence of high IgM binding to donor CD3 positive T lymphocytes would also indicate that a similar level of IgM binding would occur with autologous leucocytes and donor endothelial cells.

TABLE V
Correlating quantity of recipient IgM binding to CD3 positive
donor T lymphocytes with human kidney transplant outcome

| | No IgM | LOW IgM | HIGH IgM |
|---|---|---|---|
| | (MCF < 20) | (MCF 21-200) | (MCF > 200) |
| # of Patients | 65 | 22 | 21 |
| % Acute Rejections | 32 | 32 | *9.5 |
| Requiring Treatment | | | |
| % Graft Loss | 20 | 9.1 | *0 |
| (1 year) | | | |

MCF = Mean Channel Fluorescence
*These data when compared to No and Low level Igm are statistically significant. (p < 0.02)

Data in FIG. 15 and Table V clearly shows that the presence of low or high IgM anti-lymphocyte activity as quantitated by mean channel fluorescence (MCF) was clearly associated with significantly less rejections and less graft loss at one year. All patients in this study were given the same immunosuppressive agents.

We next wanted to confirm whether IgM protected against rejections using a murine model. In these experiments we used C57BL/6 wild type mice referred to as B6-WT mice. Hence we did cardiac transplants in healthy appearing 10 wk old male B6-WT mice recipients and compared them to B6 IgM knockouts recipients (referred to as B6 IgM ko). Donor hearts were obtained from C57BL/6 mice that were incompatible at the MHC Class II locus (referred to as B6-bm12). In these experiments, B6-WT and B6-IgM ko mice transplanted on the same day were both euthanized on day 14, the day when B6-IgM ko began demonstrating decreased cardiac contractility by finger palpation. Cardiac contractility began decreasing on day 21-28 in B6-WT mice, which has also been observed by others.

Histological evidence in B6-IgM ko mice (n=8) revealed extensive parenchymal inflammation with myocardial necrosis, edema, hemorrhage and vasculitis with endothelitis (but not with vascular occlusion) score 3.8±0.2. (see FIG. 17). At 2 weeks, in B6-WT mice (n=7), histology revealed few focal areas of parenchymal inflammation with mild necrosis but with no vasculitis or hemorrhage (score: 2.6±0.2). The parenchymal inflammation scores that we observed at two weeks in the B6-WT recipients are similar to the findings of others. Criteria used for histological scores are detailed in Research design and methods under section Method of Cardiac Transplant, Diagnosis of Rejection and Histological Score. Cardiac cessation occurred on days 14-16 for B6-IgM ko but allograft contractility was present >35 days in the B6-WT but with decreased contractility beginning after day 21. These data indicate that IgM can inhibit T cell mediated inflammation.

According to the present invention, the inventor believes that IgM anti leucocyte antibodies mediate protection against rejections by binding to autologous leucocytes (thus inhibiting chemotaxis of leucocytes and lymphocyte activation) and receptors on donor endothelial cells. The inventor has prior art clearly demonstrating that certain kidney recipients have IgM in their serum that binds to both donor lymphocytes and kidney endothelial cells. These data are described in Lobo et al, Lancet 2: 879-83, 1980.

h) IgM anti-lymphocyte antibodies cause apoptosis of Lymphoma cells at 37° C.

Malignant T lymphocytes, unlike normal IL-2 activated lymphocytes, undergo apoptosis in presence of IgM at 37° C. In these studies, we added 5 to 10 microgm of normal pooled IgM to 0.5×10⁶ Jurkat or Sup T-1 lymphocytes in 0.5 ml of RPM1 with 2% albumin. After thirty to 45 minutes incubation at 37° C. in 5% CO₂ cells were examined for apoptosis with anti-annexin antibodies and flowcytometry. No exogenous complement was added. Twenty to 35% of Jurkat or Sup T-1 cells were found to be dead under these conditions. There was less than 5% cell death of normal human lymphocytes or IL-2 activated lymphocytes when cultured under these conditions.

i) Evidence to Show that (i) Binding of IGM-ALA is Highly Specific (ii) IGM-ALA is Naturally Occurring and (iii) Naturally Occurring IgM-ALA Clones Will Inhibit HIV-1

(i) Since IgM is a pentameric molecule and is encoded by non-mutated germ line genes, it became necessary to exclude the possibility of non-specific binding by IgM to carbohydrate moeties on leucocyte receptors. This possibility was examined by evaluating the binding of IgM from a panel of IgM secreting human B cell clones derived from human umbilical cord blood. Of the 79 supernatants containing IgM (concentration 300-1100 ng/ml), only 8 had IgM secreting clones with IgM-ALA reactivity. Clones secreting IgM-ALA had the following binding specificities to cell lines: 2 clones with only anti-T cell (Jurkat, Sup T-1) reactivity, 2 clones with only anti-monocyte (U937) reactivity and 4 clones with reactivity to all cell lines i.e., T, B and U937 (monocytoid) cells (See FIG. 18A). Two of these 8 wells also had IgM that bound to soluble CD4 and CD81 as evaluated by an ELISA technique (FIG. 18B). All 8 clones secreting IgM-ALA did not bind to IgG coated latex beads. These findings i.e., presence of IgM-ALA reactivity in 10% of IgM secreting clones (and not the majority of IgM secreting clones) and binding of IgM-ALA to CD4, CD81 and specific cell lines (i.e., T cells or monocytes) would strongly argue against non-specific IgM binding to leucocyte receptors and provide in addition evidence indicating that IgM-ALA consists of different antibodies having different specificities e.g., IgM with specificity for CD4 and another antibody with specificity for a receptor on T or monocyte cells. Furthermore, binding of IgM to all cell lines i.e. T cells, Daudi B cells and U937 can best be explained either by polyspecificity of certain IgM autoantibodies or by binding of IgM-ALA to a leucocyte receptor that is common to all cell lines e.g. CD45, CD81 or chemokine receptors.

(ii) The production of IgM-ALA by umbilical cord B cells provided more evidence to show that IgM-ALA belong to the repertoire of naturally occurring IgM antibodies. We therefore evaluated umbilical cord serum to determine the quantity of IgM using an ELISA technique (Zepto Metrix, N.Y.) as well as to determine if IgM-ALA was present in serum. Of the 7 sera analyzed, IgM was detectable in 6 sera at concentrations of 30-40 μg/ml while in one serum (cord blood #7) the IgM was present at 130 μg/ml. All cord sera had IgM-ALA (FIG. 18C). Such data indicate that IgM-ALA is present in normal sera at birth.

(iii) Further studies were performed with IgM obtained from EBV transformed human B-cell clones, to more conclusively exclude the possibility of non-specific inhibition of HIV-1 by IgM and to determine if all IgM-ALA were inhibitory to HIV-1. Details on development, isolation, and characterization of these B-cell clones are described in methods section. In these studies we compared IgM with and without IgM-ALA activity. As depicted in FIG. 18D, not all IgM is inhibitory to HIV-1. In particular, IgM without ALA activity is clearly non-inhibitory, furthermore, the data would indicate that IgM-ALA with anti-CD4 specificity has more anti-HIV activity.

j) Naturally Occurring IGG-ALA are not Present in Normal Sera and in Supernatants of Umbilical Cord B Cell Clones

Prior art demonstrating the presence of naturally occurring IgG autoantibodies, with reactivity to intra-cellular proteins, prompted studies to examine if IgG-ALA are also present in newborn sera, normal sera and disease sera. In these studies, presence of IgG-ALA was evaluated by binding of IgG from B cell clone supernatants or sera to cell lines. No IgG-ALA was detected in 96 umbilical cord B cell clone supernatants, in 12 cord sera and in 107 normal adult sera. However six of 135 ESRD sera had IgG-ALA that bound to either B cells or T cells. All six of these ESRD IgG-ALA were autoantibodies when tested against autologous leucocytes but none of these sera had IgG anti-HLA reactivity when tested with immobilized HLA antigens (Class I & II) using the single HLA antigen flow beads (One Lambda, Calif.). All 6 IgG-ALA were cytolytic to leucocytes at 37° C. None of 19 HIV-1 sera had IgG-ALA. These studies clearly demonstrate that IgG-ALA are not present in newborn sera and in normal sera but occasionally present in ESRD sera.

k) Studies to Show that IgM-ALA Bind to CD81 and Inhibit Hepatitis C Infectivity of Cells

Prior art teaches that CD81 present on lymphocytes, especially B cells, and hepatocytes is an important coreceptor that allows binding and entry of the hepatitis C virus into these cells. Accordingly the inventor wanted to determine if IgM-ALA binds to CD81 especially since inventor has shown that IgM-ALA levels markedly increases in patients with chronic hepatitis C infection. In these studies presence of IgM-ALA with anti-CD81 reactivity was evaluated by binding of IgM from umbilical cord B cell clone supernatants in an ELISA using recombinant extracelluclar CD81. As depicted in FIG. 18B, two of the 41 clones had specific antibody reactivity to the extracellular domains of CD81. These two clones did not bind to CD81 with mutation in amino-acid 186, which is the binding epitope for the hepatitis C virus, indicating therefore that IgM-ALA and hepatitis C virus bind to the same epitope on the CD81 receptor. However monoclonal IgM antibodies from these two clones with anti-CD81 activity also bound to CD4 indicating therefore that the polyreactive IgM anti-CD81 cross-reacts with CD4. Other IgM anti-CD4 monoclonals did not cross-react with CD81.

Since prior art teaches that hepatitis C virus can infect activated human B cells and B cell lines, we performed in-vitro assays to determine if purified IgM, after binding to CD81 and other co-receptors for hepatitis C viral entry, could inhibit hepatitis C from infecting cells. In these studies we used hepatitis C serum from patients that had >1×10⁶ viral copies/ml and used 0.03 ml of serum to infect either 5×10⁵ pokeweed mitogen (PWM) activated peripheral blood lymphocytes in 0.5 ml culture media or 2×10⁵ Daudi B cell lines in 0.5 ml culture media. At the end of 5 to 6 days in culture, cells were analyzed for the presence of hepatitis C with quantitative RT-pcr. Purified polyclonal IgM (10 to 20 μg) and umbilical cord B cell IgM anti-CD81 clone (2 μg) inhibited viral infectivity of the cells by >97% in this assay. Inhibition of hepatitis C viral infectivity, in this assay, was not observed using purified IgM pre-absorbed with lymphocyte cell lines or with 50-75 μg of pooled normal IgG or IgG isolated from hepatitis C serum. These studies indicate that IgM inhibits hepatitis C infectivity by binding to lymphocyte coreceptors important for viral entry.

l) Studies to Show that Inhibitory Effect of IGM-ALA on HIV-1 is not Affected by Human Serum

We performed these studies to determine if the inhibitory effect of purified IgM on HIV-1 infectivity is inhibited by normal sera and therefore rendered in-effectual when administered in-vivo. As depicted in FIG. 19 normal sera did not inhibit the inhibitory effect of purified IgM on HIV-1 infectivity. However normal sera inhibited the inhibitory effect of soluble CD4 and the human anti-gp120 antibody, 2G12 on HIV-1 (See FIG. 19). Normal sera also inhibited the inhibitory effect of IVIG (100 ug/ml) on HIV-1. These findings provide an explanation as to why the 2G12 antibody (which binds to a conserved epitope on gp120) and hyperimmune IVIG, isolated from asympotomatic HIV-1 infected individuals was found not to be effective in-vivo even though these antibodies were very inhibitory to HIV-1 in-vitro. (See Trkola A et al, Nature Med, Vol. 11, p. 615-622, 2005 and Prince A, et al., Proc Natl Acad Sci, Vol. 85, p. 6944-6948, 1988).

m) Studies to Show that LPS Enhances IGM-ALA in Normal Mice Without Causing Autoimmune Disease

We performed these studies to determine if low dose LPS will enhance IgM-ALA in normal mice and secondly to determine if the increase in IgM-NAA and other antibodies after LPS will predispose to autoimmune disease. 6 week old C57BL/6 mice were injected intraperitoneally with 30 ug LPS (Esherichia Coli strain 011.B4 (Sigma)) at weekly intervals for 4 weeks. One week after the last dose of LPS, mice were euthanized and blood checked for hemoglobin levels, serum creatinine, IgM-ALA levels with autologous spleen lymphocytes, and histology (both H&E and immunofluoroscence) of kidney sections. In six mice given LPS and 4 control mice (without LPS), there was no difference in hemoglobulin levels, serum creatinine, and no disease or immune deposits were observed in kidney sections. However in all six mice, there was at least a four fold increase in IgM-ALA levels as determined by binding of serum IgM to splenic T lymphocytes. MCF of IgM binding to T lymphocytes in normal controls was 26±7 channels and in LPS treated mice was 144±23 channels. In another group of mice, IgM-ALA levels were evaluated 6 weeks after discontinuing LPS to determine if there was a decrease in IgM-ALA levels. There was no decrease in IgM-ALA levels when comparing with serum obtained at 1 week after discontinuing LPS. IgM purified from the serum of LPS treated mice (but not the IgM from control mice) inhibited (by >50%) chemotaxis of splenic lymphocytes in response to RANTES and inhibited (by >50%) T cell proliferation of autologous T cells in response to allogenic cells obtained from Balb/c mice. Spleen cells obtained from LPS treated mice did not have increased intra-cytoplasmic IL-2, INF-γ or TNF-α production in their T cells when compared to lymphocytes from control mice. In many experiments, there was less (30-50% less) intracytoplasmic IL-2 and TNF-α in lymphocytes from spleen of LPS treated mice, when compared to controls, indicating that LPS, in the doses used, did not activate T lymphocytes. Prior art has shown that in-vivo LPS will not activate T cells in both mice and human (See Lauw F N et al, Infection and Immunity, Vol. 68, 1014-1018, 2000).

### BRIEF DESCRIPTION OF THE DRAWINGS

FIG. 1A is a flow cytometry histogram depicting binding of Normal IgM, HIV IgM and AIDS IgM to Sup T-1 cells.

FIG. 1B is a graph depicting binding of Normal IgM, HIV IgM and AIDS IgM to GHOST CXCR4 cells.

FIG. 1C is a flow cytometry dot plot showing lymphocytes and neutrophils separated by size and derived from human blood.

FIG. 1D is a flow cytometry histogram depicting binding of Normal IgM to human T lymphocyte derived from peripheral blood cells.

FIG. 1E is a flow cytometry histogram depicting binding of AIDS IgM to human neutrophils derived from peripheral blood cells.

FIG. 2 is a histogram showing that Normal IgM will inhibit HIV-1 IIIB even when added to cells 4 days after HIV-1 infection of cells

FIG. 3A is a flow cytometry dot plot depicting that normal IgM will inhibit (i) HIV-1 (R5) 8658 viral strain from infecting GHOST-CCR5 (upper panels) and (ii) HIV-1 (X4) IIIB viral strains from infecting GHOST-CXCR4 (lower panels).

FIG. 3B are phase-contrast microscopic images (x200 magnification) to depict the inhibitory effect of HIV-IgM and Sol CD4 on syncytia formation after co-culturing X4-IIIB infected T cells (SupT-1 cell line) with un-infected SupT-1.

FIG. 3C are histograms depicting that normal IgM inhibits binding and entry of pseudotyped HIV-1 viral envelopes, obtained from different X4 and R5 viral strains, into activated PBL.

FIG. 4 is a bar histogram depicting that IgM, especially HIV and ESRD, inhibits proliferation of peripheral blood lymphocytes (PBL) activated in an MLR.

FIG. 5 is a bar histogram depicting that IgM, especially HIV and ESRD, inhibits proliferation of T lymphocytes activated by anti-CD3 antibody.

FIG. 6 is a bar histogram depicting that IgM, especially ESRD IgM, inhibits SDF-1α (CXCL12) induced chemotaxis of HuT 78 (upper panel) and Jurkat (lower panel) malignant T cell in as well as chemokinesis of cells (see bars shaded grey) in absence of SDF-.

FIG. 7 is a western blot to show differences in immunoprecipitation of CD3e and CD4 by normal (labeled N, 1, 2, etc.), individual HIV (labeled H) and individual ESRD (labeled E) IgM from whole cell lysates of Jurkat cells. Ly and (Ly+B) are control lanes with only lysate (Ly) or lysate mixed with bead (Ly+B) but without IgM.

FIG. 8 are flow cytometry histograms depicting that ESRD IgM inhibits membrane expression of CD4, and CD2 but not CD8 and CD28. The shaded histogram represents receptor expression (quantitated by mean channel fluorescence—MCF) in absence of IgM.

FIG. 9 is a flow cytometry histogram depicting that ESRD IgM, but not normal IgM, inhibits the co-stimulatory molecule CD86, on macrophages activated in an MLR for 24 hrs. The shaded histogram represents receptor expression in absence of IgM.

FIG. 10 depicts flowcytometry dot plots to indicate normal IgM, but not control Waldenstrom IgM, inhibits CD4 expression on cell surface of T cells activated in a 3 day MLR (left panels) as well as intracytoplasmic CD4.

FIG. 11 Panel A depicts flowcytometry dot plots to show that ESRD and Normal IgM inhibits background phos-Zap-70 (shaded grey) in PBL as well as the increase in phos-Zap-70 following 16 hours of activation with anti-CD3 (OKT3). Panel B are bar histograms to show that all the different IgM (4 different HIV, one pooled ESRD, one Normal IgM) but not control Wadenstrom IgM, inhibited the increase in phos-Zap-70 after 16 hours of anti-CD3 activation. Data also depicts total Zap-70 (shaded bars) which did not increase with anti-CD3.

FIG. 12A depicts a cytokine array where the size and density of positive spots denotes in a semi-quantitative manner, the presence of cytokines in the MLR supernatants. Note that normal, ESRD and HIV-1 IgM inhibits production of TNF- and IL-13 but not IL-6, IL-8, MIG and MCP-1.

FIG. 12B depicts flowcytometry dot plots to show that IgM inhibits production of intracytoplasmic IL-2 but not INF-γ of PBL activated for 48 hours in an MLR.

FIG. 13 are western blots depicting differences in immunoprecipitation of CXCR4 and CCR5 by individual normal IgM (labeled N 1 or 2), pooled normal IgM (labled N-P), pooled ESRD IgM (labeled E-P), individual HIV-1 IgM (labeled H 1 or 2, etc) and Waldenstrom IgM (labeled W). Lanes labeled Ly or Ly+B are similar controls as in FIG. 7.

FIG. 14 are graphs depicting that normal and ESRD IgM, IgM, inhibits binding of SDF-1α to SupT-1 cells (left panels) and MIP-1α binding to U937 cells (right panels).

FIG. 15 are flow cytometry histogram of Jurkat Cells depicting that ESRD IgM does not internalize CXCR4 (Panel A) but ESRD IgM will prevent internalization of CXCR4 receptor induced by SDF-1α (Panel B).

FIG. 16 depicts flow cytometry dot plots to show that different kidney transplant recipients have in their serum different quantities of IgM binding to their donor CD3 positive T lymphocytes. The lower dot plots depict binding of IgM to donor T lymphocytes after adding sera obtained from different recipients. Some recipient sera have no IgM anti-T lymphocyte antibody (left panel) while other sera have very high IgM anti-T lymphocyte antibody (right panel) as quantitated by mean channel fluorescence (MCF).

FIG. 17 depicts histological sections of transplanted murine hearts to show that there is more severe rejections when hearts are transplanted into IgM knockout mice.

FIG. 18A depicts binding of monoclonal IgM-ALA to leucocytes. Note that certain clones bind only to T cells (CD3+) while others bind only to macrophages.

FIG. 18B depicts binding of monoclonal IgM-ALA to soluble CD4 and soluble CD81 in an ELISA assay. Note that only a few clones bind specifically to either CD4 or CD81.

FIG. 18C depicts inhibition of HIV-1 with monoclonal IgM-ALA. Note that IgM without anti-leucocyte activity fails to inhibit HIV-1.

FIG. 19 depicts that human serum does not inhibit the inhibitory effect of IgM on HIV-1 but inhibits the inhibitory effect of soluble CD4 and 2G12 (anti-gp120) on HIV-1.

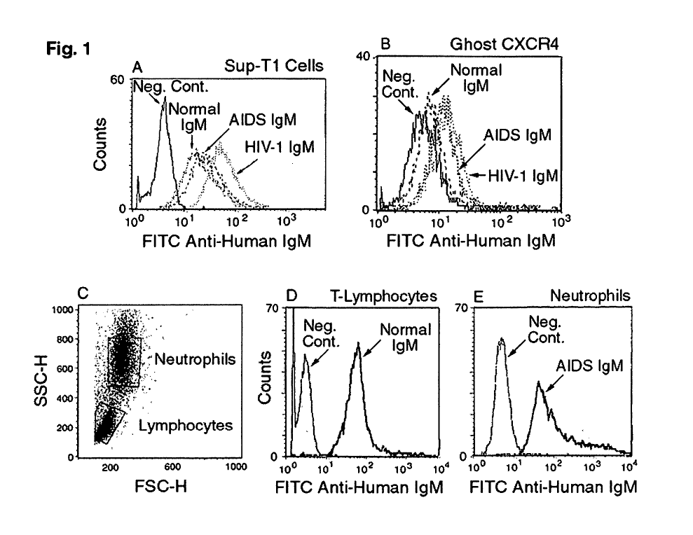

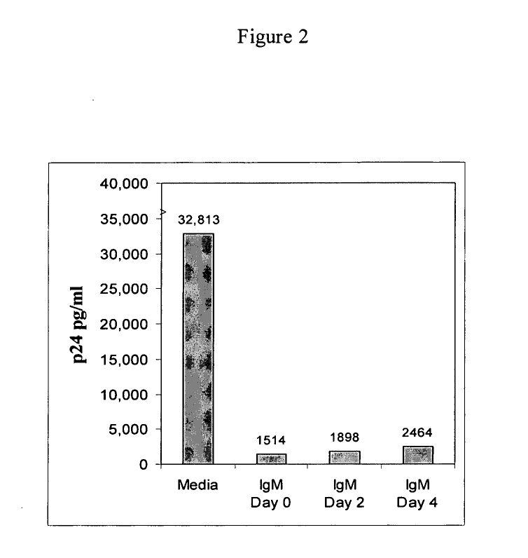

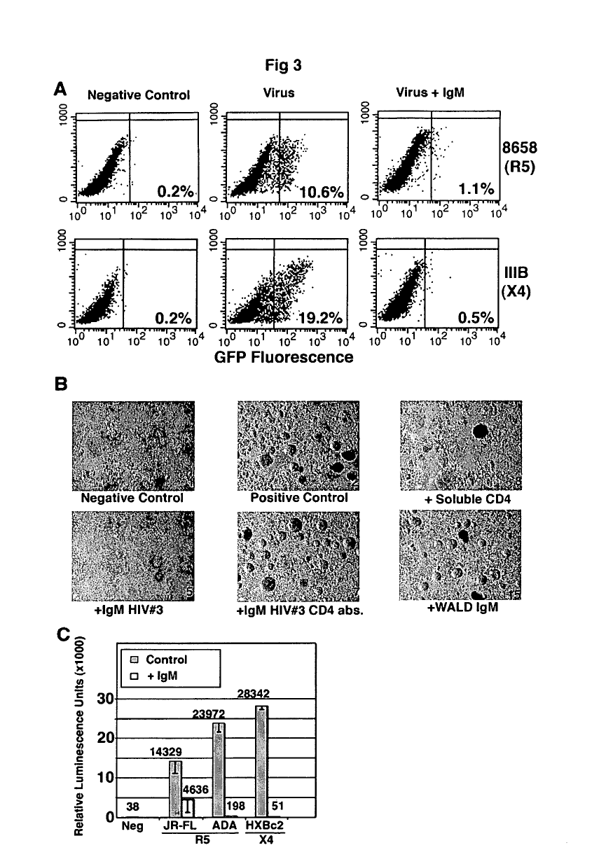

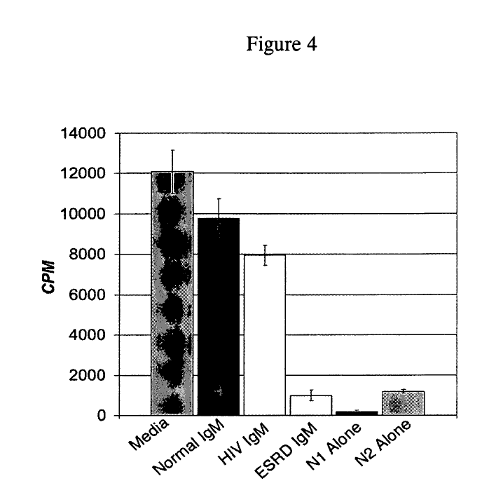

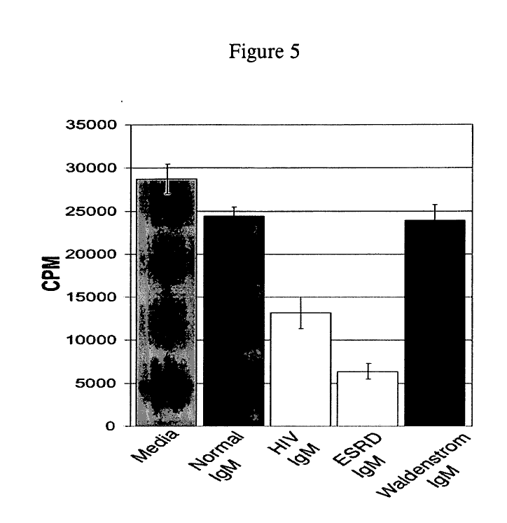

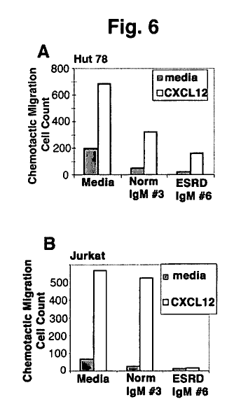

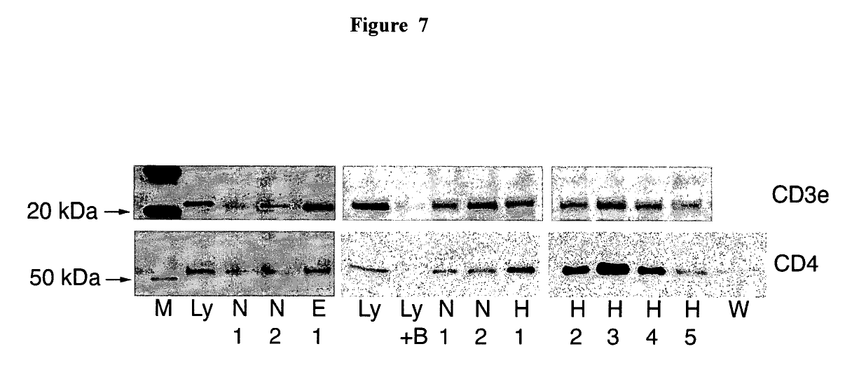

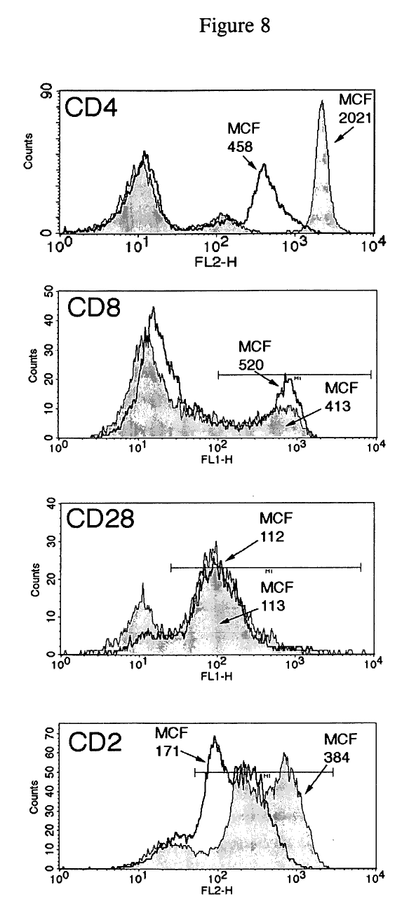

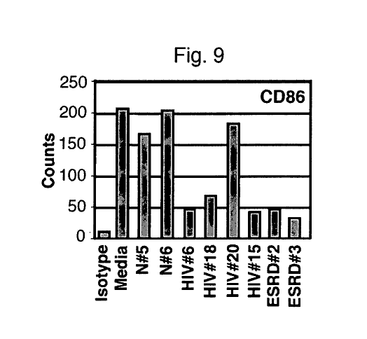

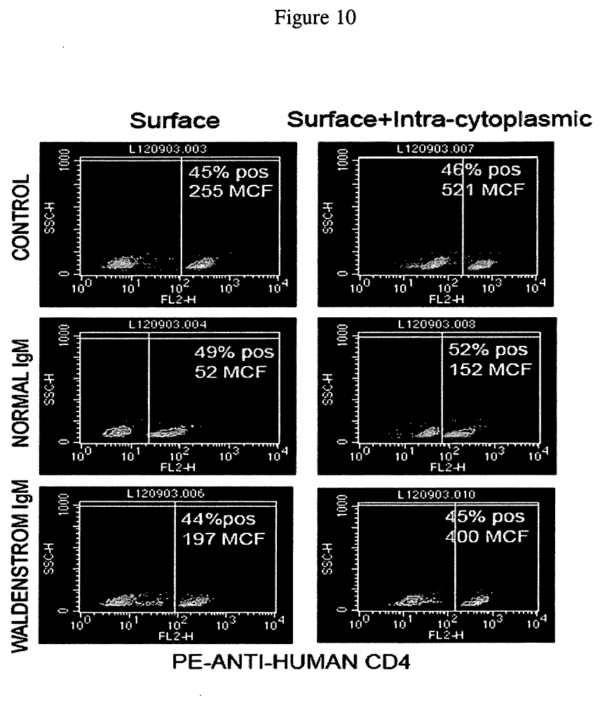

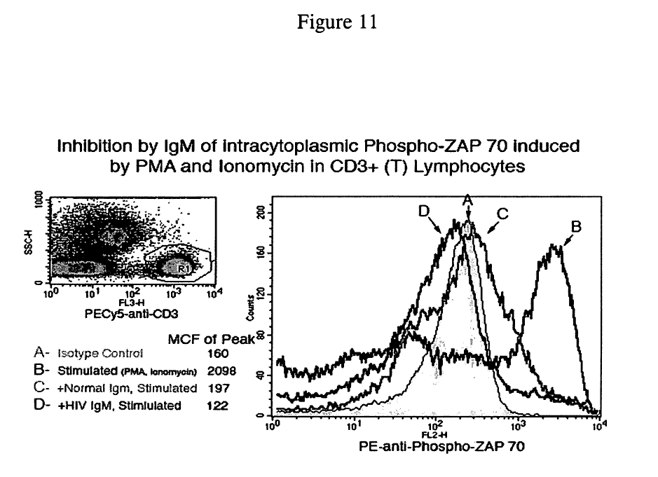

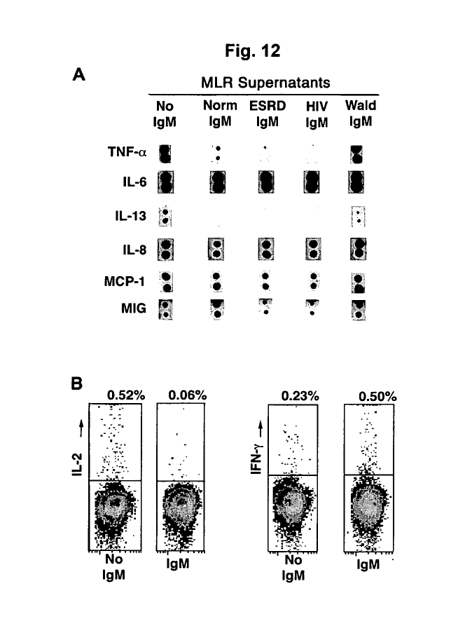

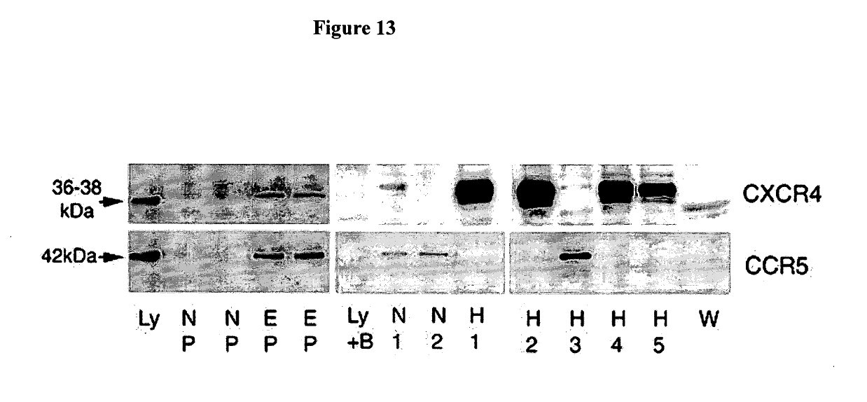

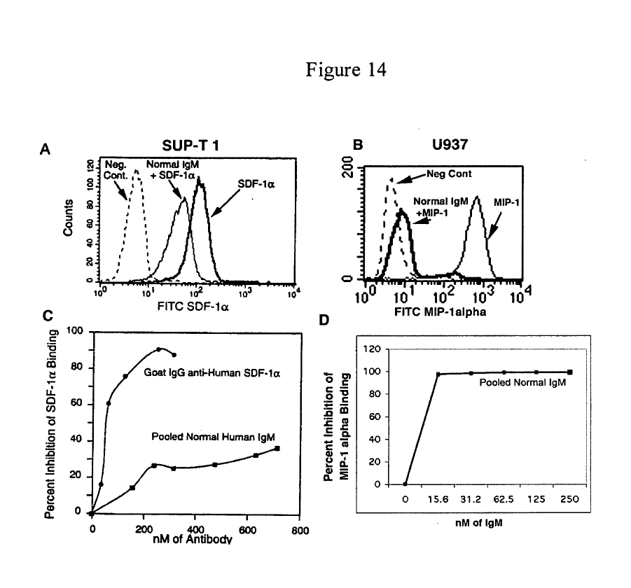

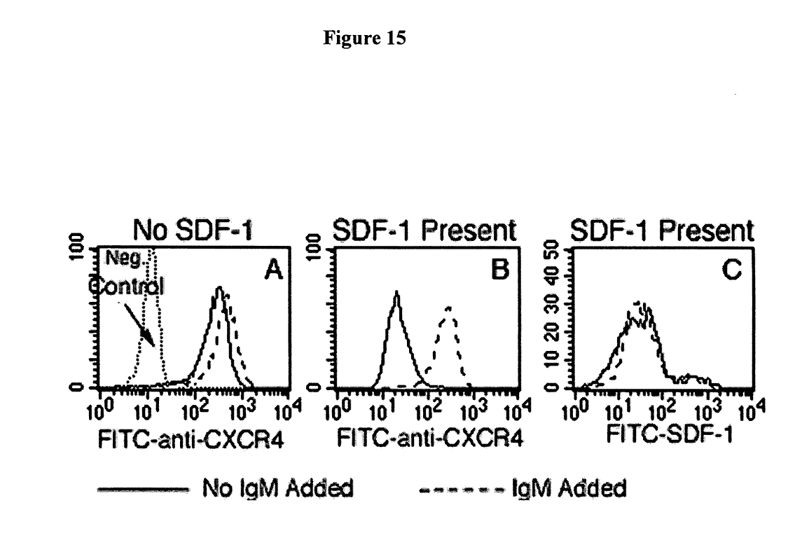

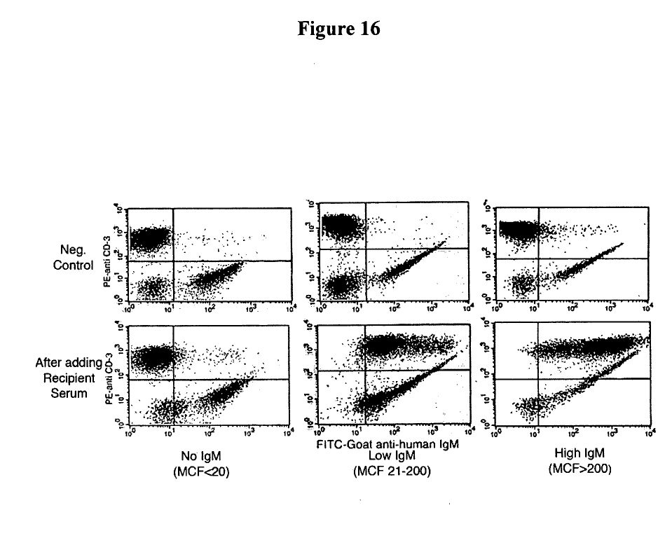

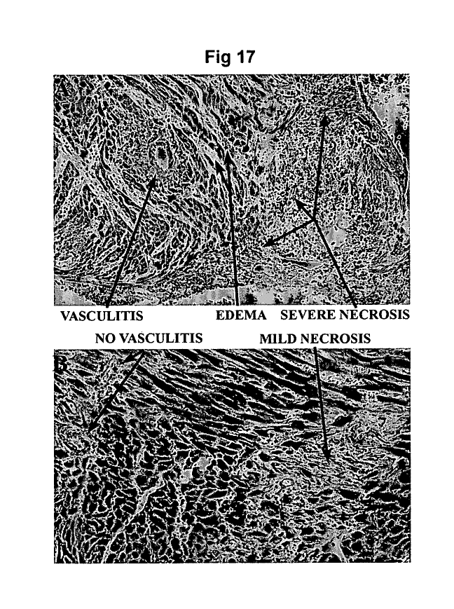

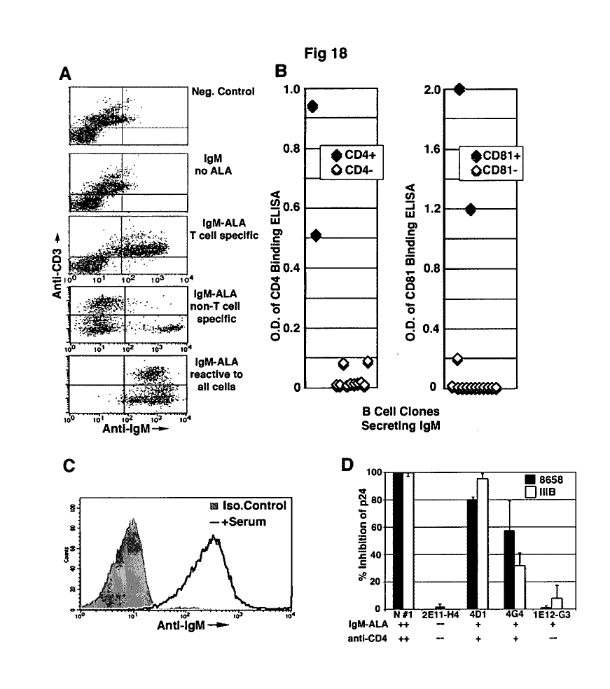

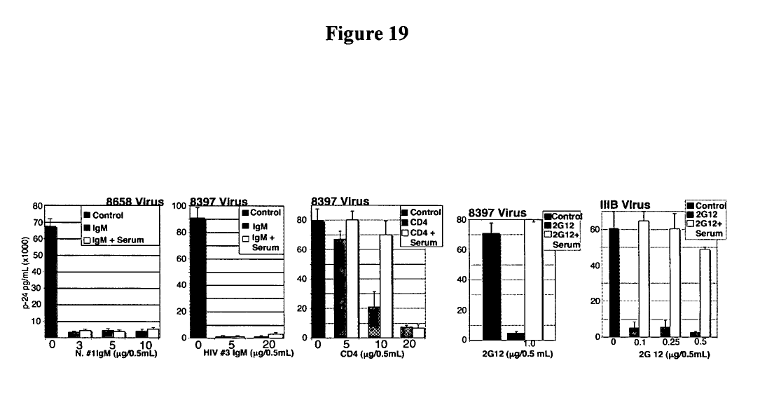

### MODES FOR CARRYING OUT INVENTION

While not wishing to be bound to any particular theory, there are several possible explanations for the entry of the HIV-1 virus into cells and increased viral replication despite the presence of a good levels of naturally occurring IgM autoantibodies to CD4 and chemokine receptor during the asymptomatic state. One such explanation is the possibility that there exists a delicate balance between these low-affinity binding IgM antibodies and the viral load. Factors that predispose an individual to an increased viral load or that inhibit the B cells secreting IgM autoantibodies will lead to viral entry into cells and to disease progression. It is also possible that the recently described subset of B cells expressing CD4, CXCR4 and CCR5 receptors may be the same subset that secretes such IgM autoantibodies. Over several months or years, this B cell subset could be exhausted or could be infected with HIV-1, thereby leading to a decrease in antibody production. Additionally, one cannot underscore the importance of other host factors (e.g., anti-viral IgG antibodies, chemokines and complement and cytotoxic T cells) that decrease the viral load. Perturbation in any of these host defense mechanisms could lead to an increased viral load.

That naturally occurring IgM autoantibodies inhibit HIV-1 and hepatitis C virus from cell entry and replication supports the premise for a protective role mediated by these IgM anti-leukocyte antibodies. The use of effective amounts of isolated human IgM anti-leukocyte antibodies to treat and reduce HIV-1 and hepatitis C infectivity (i.e., through receptor blockade and/or inactivation of cells) is an alternative approach for passive immunization to treat progressive worsening of HIV-1 infection or to treat individual's that have been exposed to HIV-1 as for example after a needle stick or after a sexual encounter. Receptor blockade by administering IgM with reactivity to a broad range of chemokine and other receptors present on the lymphocytes may be particularly useful in situations where the HIV-1 virus switches its receptor usage, e.g., from CCR5 to CXCR4. The inventor shows that polyclonal IgM is inhibitory at lower doses (approximately 5 fold less) when compared to pooled IgG, which is conventionally used as IVIG in divided doses for a total of 3 gm/kg body weight to treat different inflammatory states. The inventor believes that adequacy and frequency of IgM to be administered in-vivo can be evaluated by functional in-vitro assays quantitating the level of IgM-ALA with specific receptor binding or with inhibitory activity in blood or body fluids obtained from the individual. Maintaining increased levels of such protective antibodies e.g. through a vaccine strategy could also increase the latency period after HIV-1 infection or maintain an inflammatory state in remission.

Additionally, it may be possible to design immunization strategies or vaccines to enhance in-vitro and in-vivo IgM anti-lymphocyte NAA production by B1 lymphocytes. Enhanced production of IgM-ALA can be used to prevent or treat HIV-1 or hepatitis C as well as to treat inflammatory conditions. Strategies to enhance in-vivo or in-vitro IgM-ALA could include mitogenic antigens derived from viruses, bacteria, fungi, mycloplasma, protozoa, helminthes, or plants or esters e.g. phorbol esters. Such mitogenic antigens could be used in-vitro or for in-vivo use, administered orally, intramuscularly, intravenously or subcutaneously. Other strategies to enhance in-vitro or in-vivo IgM-ALA could include the use of cytokines, steroids, hormones or vitamins, e.g. Vitamin A or D. Prior art has shown that steroids or vitamins can influence antibody production. (See Wang W et al, Clin. Exp. Immunol 92:164-168, 1993 and Reinhardt T Z et al, J of Dairy Science 82:1904-1909, 1999). Similarly prior art has shown that cytokines and phorbol esters can preferentially stimulate B1 cells to enhance production of IgM-NAA (See Baumgarth N., et. al., Springer Semin. Immuno, 2005, Vol. 26, p. 347-362).

There is concern that strategies aimed at polyclonal stimulation of B-1 cells could cause auto-immune disease. The inventor believes that polyclonal stimulation of B-1 cells in normal individuals or animals and in absence of immune dysregulation induced genetically or secondary to environmental factors as for example in SLE, is rarely a cause of autoimmune disease. Stahl D, et. al. teaches (See J of Immunol Methods, Vol. 240, p. 1-14, 2000) that in SLE, hemolytic anaemia mediated by IgG autoantibodies is a result of deficiency of IgM-NAA directed to the variable region of IgG anti-RBC antibodies (i.e. there is dysregulation in anti-idiotypic antibodies). Baumgarth N et al (See Springer Semin Immunol, Vol. 26, p. 347-362, 2005) teaches that unbridled antibody production by B-1 cells could result in serious autoimmune problems. The main evidence for the teachings of Baumgarth come from in-vivo studies where after LPS, mice genetically modified with a transgene to encode IgM anti-red blood cell (RBC) antibodies developed hemolytic anaemia resulting from excess IgM anti-RBC antibody production in these transgeneic mice. The inventor objects to the teachings of Baumgarth for two main reasons. Firstly, the authors who performed the LPS experiments on autoantibody transgenic mice (Murakami et al, J of Exp Med, Vol. 180, p. 111-121, 1994) in a later article (See Murakami et al, Immunol Today, Vol. 16, p. 5340538, 1995) teach the following “in spite of the lessons from anti-RBC-Tg mice and other circumstantial evidence for the involvement of B-1 cells in autoimmunity, the question remains open as the whether B-1 cells produce the pathogenic autoantibodies that are involved in auto-immune disease”. Secondly, in the current application, the inventor shows that LPS when injected into normal mice, enhances production of IgM-ALA without causing auto-immune disease.

The inventor believes that IgM-ALA, by binding to coreceptors for hepatitis C viral entry can also inhibit progression of hepatitis C and development of acute and chronic liver disease. Hence similar strategies for the treatment and prevention of HIV-1 can also be applied for hepatitis C including strategies that will enhance IgM-ALA in-vitro and in-vivo.

Diseases associated with tissue-specific inflammatory processes, angiogenesis and growth (and spread) of malignant cells are controlled by chemokines, cytokines, chemokine receptors and other receptors that activate (or inhibit) cell function. Such receptors are present on all leucocytes, endothelial cells and malignant cells. IgM anti-lymphocyte NAA, by binding to chemokine and other receptors (e.g. lipid rafts, CD4 and CD3) could provide a regulatory role in the above-mentioned disorders or processes. The use of isolated IgM anti-lymphocyte NAA, especially antibodies that inhibit chemokine receptor function or inhibit cell activation (i.e. with potential of causing apoptosis of malignant cells) or production of pro-inflammatory cytokines especially TNF- would be particularly beneficial to treat inflammatory processes or growth and spread of malignant cells or to prevent and treat allograft rejection. Studies in renal transplant recipients clearly indicate that chemokines and chemokine receptors have a role in the rejection process. Data in this regard is reviewed in Hancock, W. W, J of Am Soc Nephrol 13: 821-824, 2002. Hence, the finding that kidney transplant recipients, with low or high levels of IgM anti lymphocyte antibodies, have no or minimal acute rejections would support the concept that IgM anti-lymphocyte antibodies inhibit chemokine receptor function and lymphocyte activation. One could employ passive immunization technique or alternatively design immunization strategies (described above) to specifically enhance in-vivo production of IgM anti-lymphocyte NAA (with inhibitory effect on entry of pathogens e.g., HIV-1, Hepatitis C or malaria, or chemokine receptor function or cell activation) to treat the various infective conditions, inflammatory processes and growth (and spread) of malignant cells. The effective dose and frequency of IgM administered for passive immunization in-vivo can be determined by functional assays to quantify in-vivo levels of IgM-ALA with the desired receptor specificity (as described below). The inventor further believes that vaccine strategies to increase IgM-ALA will not be used for autoimmune inflammatory disorders caused by IgG high affinity binding autoantibodies e.g. in SLE, as such strategies can increase production of the pathogenic autoantibodies. Hence in claims the term inflammatory states will be used to denote inflammation that is not mediated by an autoimmune disorder involving pathogenic autoantibodies. Conversely in claims, the term autoimmune disease/disorder will denote inflammation mediated by pathogenic autoantibodies that are mostly of IgG isotype, but could also involve antibodies of IgM and IgA isotypes.

The source of naturally occurring IgM antibodies may be heterologous, autologous or allogeneic. Naturally occurring IgM antibodies with specificity for chemokine and other receptors on the lymphocyte may be raised in vivo (i.e., in mice or other animals or in humans) or in vitro using cell culture techniques. Naturally occurring IgM-ALA can be used either in a preparation that is polyclonal, monoclonal or in a monomeric form.

Naturally occurring IgM antibodies with specificity for lymphocyte receptors may be produced either in vivo or in vitro. One cam employ methods of genetic engineering whereby genes specific for IgM anti-lymphocyte antibodies are introduced into antibody-producing cells. These antibody-producing cells may then be introduced into an infected human or into immunodeficient animals where the cells produce IgM antibodies with specificity for lymphocyte receptors. In the alternative, these antibody-producing cells may be grown in vitro using hybridoma or other technology.

Naturally occurring IgM antibodies with specificity for lymphocyte receptors may also be produced by isolating human or animal antibody-producing B-1 cells specific for IgM anti-lymphocyte antibodies and enhancing antibody production by such cells using hybridoma or other technology, including introduction of the cells into animals or humans. B-1 cells can be isolated from blood, spleen, bone-marrow and umbilical cord. Autologous B-1 cells, activated to enhance production of IgM-ALA can then be re-introduced into patients to treat HIV-1 or Hepatitis C viral disease or inflammatory disorders. Alternatively IgM-ALA produced in-vitro by isolated B-1 cells can be administered (intra-peritoneally, intramuscularly or intravenously) into patients to treat hepatitis C, HIV-1 or inflammatory states.

Another method of producing naturally occurring IgM antibodies is by isolating human antibody-producing B-1 cells capable of generating human IgM with specificity for lymphocyte receptors from animals such as, for example, the XenoMouse™. IgM antibody production by such B-1 cells may then be enhanced in vitro employing hybridoma or other technology such as, for example, stimulating the isolated lymphocytes with LPS or other agent that will activate the cells, e.g., the EBV virus.

Naturally occurring IgM antibodies with specificity for lymphocyte receptors may also be produced in vitro by isolating lymphocytes that can then be transformed with the EBV virus and introduced in a culture. A subset of these EBV transformed B lymphocytes will secrete IgM antibodies such that the resulting culture fluid contains these antibodies.

In addition, self antigens or antigens that are mitogenic and obtained from viruses, bacteria, fungii, protozoa, and other mitogens including plant mitogens, cytokines, esters e.g. phorbal esters, hormones and vitamins including vitamin A and D may be used to stimulate B-1 cells in vivo or in vitro to enhance generation of naturally occurring IgM antibodies to leukocytes.

IgM antibodies produced outside an infected individual may be delivered to the individual by one of several routes of administration including, but not limited to, intravenous, intraperitoneal and intramuscular delivery.

IgG, IgD, IgE and IgA isotypes of naturally occurring autoantibodies (i.e. NAA) have also been described in prior art. The present invention also relates to IgG, IgD, IgE and IgA isotypes especially since there is prior art describing the successful use of a technology for the molecular cloning of combinatorial phage display libraries containing genes coding exclusively for antibody fragment of the IgM, IgD, IgA or IgG phenotype as well as genes from a naturally expressed human antibody repertoire. (See Raum T, Cancer Immunology, Immunotherapy 2001, vol. 50, p. 141-50, Burioni R, Research in Virology 1998, vol. 149, p. 321-25). In this prior art human natural antibodies of the IgM phenotype can through this technology, be switched to another phenotype. All antibody isotypes (i.e. IgM, IgE, IgG and IgA) in this invention includes intact immunoglobulins or fragments of these antibodies. As such, throughout the specification and claims the use of the term “antibodies” or auto antibodies” includes naturally occurring antibodies of all isotypes used as intact immunoglobulins or fragments of these antibodies.

This invention can be used to develop an in-vitro method for quantifying in-vivo levels of IgM-ALA with different receptor specificities (including epitope specificities) in disease states or in individual's at risk for being infected with HIV-1 or Hepatitis C or after administration of purified IgM or after vaccine therapy to enhance production of IgM-ALA. For example one can test blood, serum, other body fluids or purified IgM from a patient with an inflammatory disorder and determine if there is adequate IgM-ALA that will effectively inhibit or enhance chemotaxis, T-cell activation/proliferation in response to autologous or allogeneic leucocytes or mitogens. Similarly this invention can be used to develop an in-vitro method to test blood, serum, other body fluids or purified IgM from a patient infected with HIV-1, Hepatitis C or malaria and determine if there is adequate IgM-ALA that will effectively inhibit viral or protozoal entry into autologous or allogenic cells or binding of these viruses and viral envelopes on autologous or allogenic cells. Individual's at risk could include individual's who are at risk of being exposed (or have been exposed) to an infective agent e.g. HIV-1 or individual's who could develop an inflammatory state e.g. after an allograft transplant. Prior art has developed IgG monoclonal antibodies specific for the binding epitopes on coreceptors important for binding of HIV-1 and Hepatitis C viruses. This current invention can be used to develop an in-vitro method to determine if IgM from patient will inhibit binding of these IgG anti-coreceptor monoclonal antibodies. In this way, one can determine if an individual's IgM-ALA has antibodies specific for viral binding epitopes on the coreceptors. Such assays will be particularly useful as for example both AIDS and HIV-1 patients have high levels of IgM-ALA, but AIDS IgM-ALA is ineffective in inhibiting HIV-1 viral entry of several viral isolates.

Having now fully described the invention with reference to certain representative embodiments and details, it will be apparent to one of ordinary skill in the art that changes and modifications can be made thereto without departing from the spirit or scope of the invention as set forth herein.

The material in the references listed below is herein incorporated in this application to provide more detailed information that will enable the claims.

### REFERENCES

Banapour B, Sernatinger J, and Levy J. B (1986) The AIDS-associated retrovirus is not sensitive to lysis or inactivation by human serum. Virology, 152, 268-271.
Barel M, et al. (2003) Activation of EBV virus/C3d receptor (gp140), CR2, CD21) on human cell surface triggers pp60src and Akt-GSK3 activities upstream and downstream to PI3-kinase, respectively. European J Immunol 33, 2557-2566.
Berberian L, Goodglick K, Kipps T J, Braun J. (1993) Immunoglobulin VH3 gene products: natural ligands for HIV gp120. Science 261 September 17; 1588-91.
Baumgarth N, et al (2005) Inherent specificities in natural antibodies: a key to immune defense against pathogen invasion. Springer Semin Immuno, 26, 347-362.
Boes M, et al (2000) Role of natural and immune IgM antibodies in immune responses Molecular Immunol, 37, 1141-1149.
Cervenak J, Kiss K, & Uher F (1999) Partial characterization of two lymphocyte specific natural autoantibodies isolated from new born mice. Acta Microbiologica et Immunologica Hungarica, 53-62.
Chaudhari U, et al. (2001) Efficacy and safety of infliximab monotherapy for plaque-type psoriasis: a randomized trial. Lancet, 357, 1842-1847.
Choudhry V, et al (2006) Antibody-based inhibitors of HIV infection. Expert Opin Biol Ther, 6, 523-531.
Chong T W, et al (2006) Primary human hepatocytes in spheroid formation to study hepatitis C infection. J of Surgical Research 130, 52-57.
Dimitrov D S (2004) Virus entry: molecular mechanisms and biomedical applications. Nature Reviews (Microbiology) 2, 109-121.
Feldman M, et al. (2001) Anti-TNF-α therapy of rheumatoid arthritis. Annual Rev Immunol 10, 163-96.
Frenkel, N., Schirmer, E. C., Katsafanas, G., & June, C. H. (1990) T-cell activation is required for efficient replication of human herpesvirus 6. J. Virol., 64, 4598-4602.
George J, et al. (1997) The emerging concept of pathogenic natural autoantibodies. Human Immunol, 8, 70-75.
Gerard, C. & Rollins, B. J. (2001) Chemokines and disease. Nat. Immunol., 2, 108-115.
Griggi T, Bauer R, Garofalo T, Kukel S, Lenti L, Massetti A P, Muller C, Sorice M, & Pointieri G M (1994) Autoantibodies against ganglioside GM3 represents a portion of antilymphocytic antibodies in AIDS patients. Scan J of Immunol, 40, 77-82.
Hancock, W. W. (2002) Chemokines and transplant immunobiology. J. Am. Soc. Nephrol., 13, 821-824.
Hansson, G. K., Libby, P., Schonbeck, U., & Yan, Z. Q. (2002) Innate and adaptive immunity in the pathogenesis of atherosclerosis. Circ. Res., 91, 281-291.
Hoshino H, Tanaka H, Miwa M, Okada H., 1984 Human T-cell leukaemia virus is not lysed by human serum. Nature, 310, 324-325.
Huber, B. T., Hsu, P. N., & Sutkowski, N. (1996) Virus-encoded superantigens. Microbiol. Rev., 60, 473-482.
Hurez V, et al. (1994) Anti-CD4 activity of normal human immunoglobulin G for therapeutic use (Intravenous Immunoglobulin, IVIG). Therapeutic Immunology. 1, 269-277.
Jenkins, M. K., Khoruts, A., Ingulli, E., Mueller, D. L., McSorley, S. J., Reinhardt, R. L., Itano, A., & Pape, K. A. (2001) In vivo activation of antigen-specific CD4 T cells. Annu. Rev. Immunol., 19, 23-45.
Julien S, et al (2002) B cell positive selection by soluble self antigen. J of Immunol, 169, 4198-4204.
Lacroix-Desmazes S, Kaveri S V, Mouthon L, Ayouba A, Melanchere E, Coutinho A, & Kazatchkine M D (1998) Self-reactive antibodies (natural autoantibodies) in healthy individuals. J of Immunol Methods, 216, 117-137.
Lauw F et al (2000) Reduced Th1, but not Th2, cytokine production by lymphocytes after in-vivo exposure of healthy subjects to endotoxin. Infection and Immunity, 68, 1014-1018.
Llorente M. Sanchez-Palomion S, Manes S, Lucas, P, Kremer K, De Alboran I M, Joran J L, Alcami J, Del Real G, Martizne-A C. (1999) Natural human antibodies retrieved by phage display libraries from healthy donors: polyreactiveity and recognition of human immunodeficiency virus type 1 gp 120 epitopes. Scand J Immunol., 50, 270-279.
Lobo P I, Westervelt F B, Rudolf L, & White C (1980) Cold lymphocytotoxins-an important cause of acute tubular necrosis (ATN) occurring immediately post-transplant. Lancet, 2, 879-882.
Lobo P I, et al. (1995) The use of pronase digested human leucocytes to improve specificity of the flowcytometric crossmatch. Transplant International, 8 472-480.
Lobo P I (1981) Nature of autolymphocytotoxins present in hemodialysis patients. Transplantation, 32 233-237.
Lobo P I (1978) Kidney transplantation across positive B and T cell crossmatches. Transplantation, 26, 84-86.
McDevitt, H., et al (2002) Multiple roles for tumor necrosis factor-alpha and lymphotoxin alpha/beta in immunity and autoimmunity. Arthritis Res, 4 (Supp 31), S141-S152.
Muller, A., Homey, B., Soto, H., Ge, N., Catron, D., Buchanan, M. E., McClanahan, T., Murphy, E., Yuan, W., Wagner, S. N., Barrera, J. L., Mohar, A., Verastegui, E., & Zlotnik, A. (2001) Involvement of chemokine receptors in breast cancer metastasis. Nature, 410, 50-56.
Murakami M., et al (1994) Oral administration of LPS activates B-1 cells in the peritoneal cavity and lamina propria of the gut and induces autoimmune symptoms in an autoantibody transgenic mouse. J of Exp Med, 180, 111-121.
Murakami M and Honjo T. (1995) Involvement of B-1 cells in muscosal immunity and autoimmunity. Immunol Today, 16, 534-539.
Nakamura, M., Burastero, S. E., Ueki, Y., Larrick, J. W., Notkins, A. L., & Casali, P. (1988) Probing the normal and autoimmune B cell repertoire with Epstein-Barr virus. Frequency of B cells producing monoreactive high affinity autoantibodies in patients with Hashimoto's disease and systemic lupus erythematosus. J. Immunol., 141, 4165-4172.
Naparstek Y, Plotz P H (1993) The role of autoantibodies in autoimmune disease. Annual Rev Immunol 11, 79-104.
Olson, T. S. & Ley, K. (2002) Chemokines and chemokine receptors in leukocyte trafficking. Am. J. Physiol Regul. Integr. Comp Physiol, 283, R7-28.
Onuffer, J. J. & Horuk, R. (2002) Chemokines, chemokine receptors and small-molecule antagonists: recent developments. Trends Pharmacol. Sci., 23, 459-467.
Perkins, D. L. (1998) T-cell activation in autoimmune and inflammatory diseases. Curr. Opin. Nephrol. Hypertens., 7, 297-303.
Prince A, et al. (1988) Failure of a human immunodeficiency virus (HIV) immune globulin to protect chimpanzees against experimental challenge with HIV-1. Proc Natl Acad Sci USA 85, 6944-6948.
Pullar C E, Morris P J, Wood K J (2003) Altered proximal T-cell receptor signaling evens in mouse CD4+ T cells in the presence of anti-CD4 monoclonal antibodies: evidence for reduced phosphorylation of Zap-70 and LAT. Scand J Immunol., 57, 333-341.
Reinhardt T A, et al. (1999) 1,25 Dihydroxyvitamin D3 enhances milk antibody titers to E. coli J5 vaccine. J of Dairy Science 82, 1904-1909.
Rodman T C et al. (2001) Circulating natural IgM antibodies and their corresponding human cord blood cell-derived Mabs specifically combat the Tat protein of HIV-1 Exptl Hematology, 29, 1004-1009.
Rodman T C, Pruslin F H, To S E, and Winston R, (1992) Human immunodeficiency virus (HIV) Tat reactive antibodies present in normal HIV-negative sera and depleted in HIV-positive sera. Identification of the epitope J. Exp. Med., 175: 1247-1253.
Romer J et al. (1982) Characterization of various immunoglobulin preparations for intravenous application. Vox Sanguinis 42, 62-73.
Sandborn W J and Hanauer S B (1999) Anti-tumor necrosis factor therapy for inflammatory bowel disease: a review of agents, pharmacology, clinical results and safety. Inflamm Bowel Diseases 5, 119-133.
Stahl D and Sibrowski W, (2003). Regulation of the immune response by Natural IgM: Lessons from warm autoimmune hemolytic anaemiar Current Pharmaceutical Design, 9, 1871-1880.
Stahl D et al (2000) Analysis of human self-reactive antibody repertoires by quantitative immunoblotting, J of Immunol Methods, 240, 1-14.
Stein B. S. & Engleman, E. G. (1991) Mechanism of HIV-1 entry into CD4+ T cells. Adv. Exp. Med. Biol., 300, 71-86.
Stimmler M. M., Quismorio, F. P. J., McGehee, W. G., Boylen, T., & Sharma, O. P. (1989) Anticardiolipin antibodies in acquired immunodeficiency syndrome. [see comments]. Archives of Internal Medicine, 149, 1833-1835.
Sutkowski N., Conrad, B., Thorley-Lawson, D. A., & Huber, B. T. (2001) Epstein-Barr virus transactivates the human endogenous retrovirus HERV-K18 that encodes a superantigen. Immunity., 15, 579-589.
Thompson K M et al (1992) Human monoclonal antibodies specific for blood group antigens demonstrate multi specific properties characteristics of natural autoantibodies. Immunology 76, 146-157.
Torbett B., Picchio G R, & Mosier D E (1991) hu-PBL-SCID mice: a model for human immune function, AIDS and lymphomagenesis. Immunol Reviews, 124, 139-164.
Trkola A., et al. (2005) Delay of HIV-1 rebound after cessation of antiretroviral therapy through passive transfer of human neutralizing antibodies. Nat Med, 11, 615-622.
Wang W, et al (1993) The effects of retinol on in-vitro immunoglobulin synthesis by cord blood and adult peripheral blood mononuclear cells. Clin Exp. Immunol. 92, 164-168.
Werlen, G. & Palmer, E. (2002) The T-cell receptor signalosome: a dynamic structure with expanding complexity. Curr. Opin. Immunol., 14, 299-305.
Yang X et al (2001) Improved elicitation of neutralizing antibodies against primary human immunodeficiency viruses by soluble stabilized envelope glycoprotein trimers. J of Virology, 75, 1165-1171.

What is claimed is:

1. A method of treating human diseases or disorders, comprising administering to the individual an effective amount of isolated IgM containing naturally occurring anti-lymphocyte antibodies (IgM-ALA) or fragments thereof or cells producing IgM-ALA or enhancing in-vivo production of IgM-ALA.

2. The method of claim 1, where in the IgM-ALA bind to lymphocyte receptors and selected from the group of receptors that activate or inhibit cell function (or processes) or enhance death of cells or inhibit viral infectivity or inhibit chemotaxis of cells.

3. The method of claim 2, where in the cell surface receptor is selected from the group consisting of CD3, CD4, CD81, CD2, CXCR4 and CCR5 receptor or lipid raft or phosopholipids on the cell membranes or other receptors that bind to IgM-ALA.

4. The method of claim 1, wherein the polyclonal and polyreactive IgM-ALA bind to chemokine and non-chemokine receptors that are present on lymphocytes and receptors present on non-lymphocyte leucocytes, hepatocytes, fibrocytes, endothelial cells, cells from other organs or malignant cells and wherein the IgM-NAA has specificity to cell surface receptors present on lymphocytes.

5. The method of claim 4, wherein the IgM-ALA having specificity to cell surface receptors present on lymphocytes are selected from the group consisting of human, and animal, naturally occurring IgM antibodies and other naturally occurring antibodies of other isotypes that have surface receptor specificity as IgM-ALA.

6. The method of claim 1, wherein the IgM-ALA can be selected from pentameric or monomeric IgM, monoclonal or polyclonal IgM, synthetic or recombinant IgM-ALA or antibody fragments of IgM having specificity to cell surface receptors present on lymphocytes.

7. The method of claim 1, wherein the human disease or disorder, comprises virus mediated disease, autoimmune disease, inflammatory states, autoimmune disorders and cellular malignancies.

8. The method of claim 7 wherein the viral mediated disease caused by HIV-1, Hepatitis C or any one or more of the other viruses infecting lymphocytes or other cells expressing lymphocyte receptors and wherein these viruses use for cell entry chemokine or non-chemokine receptors present on lymphocytes or other cells expressing lymphocyte receptors and wherein virus cell entry and/or replication is enhanced by T cell activation and wherein viral cell entry and/or replication is inhibited by IgM-ALA that inhibits T cell activation and/or proliferation.

9. The method of claim 7 wherein the autoimmune disease is selected from the group of systemic lupus erythematosus, multiple sclerosis, and other autoimmune conditions in which the autoimmune inflammatory process is mediated by pathogenic autoantibodies and mediated by T cell activation, cytokines and chemokine receptors and wherein the IgM-ALA with binding specificity to chemokine and non-chemokine receptors will inhibit the autoimmune inflammatory process.

10. The method of claim 7 wherein the inflammatory state is selected from the group of asthma, sarcoidosis, atherogenesis and atherosclerosis, pulmonary, renal and bowel inflammatory disorders or allograft and xenograft rejections in which the inflammatory process is mediated by T cell activation, cytokines and chemokine receptors and wherein IgM-ALA with binding specificity to chemokine and non-chemokine receptors will inhibit the inflammatory process.

11. The method of claim 7 wherein the cellular malignancy involves lymphoid or non-lymphoid malignancies and wherein IgM-ALA bind to lymphocyte receptors that are also present on malignant cells and wherein IgM-ALA inhibits activation of cells, inhibits cell proliferation and enhances apoptosis of tumor cells.

12. The method of claim 1, wherein therapy would comprise use of effective amount of IgM-ALA to inhibit progression of disease processes or prevent disease processes.

13. The method of claim 8, wherein IgM-ALA binds to cell surface receptors important in inhibiting T cell activation and inhibiting viral infectivity of cells and wherein such viruses include HIV-1, Hepatitis C, EBV, CMV, Rabies virus, Herpes virus 6, influenza virus, measles and Ebola virus.

14. The method of claim 1, wherein the IgM-ALA are administered to the individual by oral routes, by subcutaneous routes, intravenously, intraperitoneally or intramuscularly.

15. A method of producing IgM-ALA in-vitro and in-vivo to treat human diseases or disorders.

16. The method of claim 15 wherein IgM-ALA are produced to treat human diseases or disorders, comprising introducing genes specific for IgM-ALA into antibody-producing cells and producing the anti-lymphocyte NAA antibodies in vitro or in vivo.

17. The method of claim 15, wherein animal or human IgM-ALA are produced to treat human diseases or disorders, comprising isolating human or animal antibody producing cells and enhancing production of IgM-ALA in-vitro.

18. The method of claim 15, wherein IgM-ALA production comprises isolating human antibody-producing cells from animals capable of generating human IgM-ALA and enhancing production of IgM-ALA in vitro or in vivo.

19. The method of claim 15, wherein the production of IgM-ALA by the antibody-producing cells is enhanced using hybridoma technology or cell culture techniques.

20. The method of claim 15, wherein the production of IgM-ALA by the antibody-producing cells is enhanced in-vitro using viruses, bacteria, antigens, mitogens, hormones, steroids, esters or vitamins.

21. The method of claim 15, wherein enhancement of IgM-ALA production in-vivo comprises administering to one or more individuals, one or more elected from the group consisting of viruses, inactive bacteria, viral and bacterial products, fungal products, plant antigens, mitogens, steroids, hormones and vitamins, and wherein the IgM-ALA antibodies are used to treat viral infections, inflammatory states and cellular malignancies.

22. The method of claims 19 and 20 wherein autologous IgM-ALA producing cells are re-introduced into an individual to treat virus mediated disease, inflammatory states and cellular malignancies.

23. The method of claims 1 and 15 for treating virus mediated disease, inflammatory states and cellular malignancies in an individual comprising administering to the individual effective amounts of IgM-ALA or enhancing in-vivo production of IgM-ALA.

24. The method of claims 1 and 15 for treating human autoimmune disease in an individual comprising administering to the individual effective amounts of IgM-ALA.

25. A method for testing adequacy of IgM-ALA levels comprising detecting and quantifying IgM antibodies, having specificity to extracellular receptors present on lymphocytes.

26. The method of claim 25 wherein the assay involves binding of individual's IgM to isolated or recombinant lymphocyte receptors or inhibition by IgM of chemokine binding to receptors or inhibition by IgM of chemotaxis or inhibition by IgM of HIV-1 or Hepatitis C viral cell infection or the binding of HIV-1 or Hepatitis C or the viral envelope to cell receptors.

27. The method of claim 25 wherein the assay involves inhibition by IgM of antibodies defined by their lymphocyte receptor specificities or their receptor epitope specificity.

28. The method of claim 25 wherein the IgM antibody is present in serum, body fluids or culture supernatants.
